(19) United States
(12) Patent Application Publication
(10) Pub. No.: US 2025/0011421 A1
(43) Pub. Date: Jan. 9, 2025

(54) ANTIBODY AGAINST HUMAN TIGIT AND USE THEREOF

(71) Applicant: GUANGDONG ANNPO BIOTECHNOLOGY INC., Guangzhou (CN)

(72) Inventors: Xiaofei WANG, Foshan (CN); Caiguo YE, Foshan (CN); Min ZENG, Foshan (CN)

(21) Appl. No.: 18/708,677

(22) PCT Filed: Dec. 8, 2022
(86) PCT No.: PCT/CN2022/137649

§ 371 Date: May 9, 2024

(30) Foreign Application Priority Data
Dec. 21, 2021 (CN) 202111572836.5
Oct. 26, 2022 (CN) 202211317991.7

(51) Int. Cl. C07K 16/28; A61K 39/00; A61K 47/68; A61P 35/00; G01N 33/68

(52) U.S. Cl. CPC C07K 16/2803; A61K 47/6849; A61P 35/00; G01N 33/68; A61K 2039/505; C07K 2317/24; C07K 2317/33; C07K 2317/732; C07K 2317/734; C07K 2317/92; G01N 2333/70503

(57) ABSTRACT

An antibody against human TIGIT or an antigen-binding fragment thereof has strong binding ability to hTIGIT is disclosed. It can induce B-hTIGIT transgenic mice T cells, jurkat cells and human PBMC cells to secrete cytokines, has a significant ADCC effect and CDC effect, can significantly reduce the tumor volume/weight in tumor bearing mice implanted with a colon cancer cell line CT26 and tumor bearing mice implanted with a breast cancer cell line 4T1 without affecting the body weights of the mice, that is, without side effects, and prolong the survival time of mice, and has good pharmacokinetic properties, and can be used for detecting the expression level of hTIGIT and treating tumors.

### CROSS-REFERENCE TO RELATED APPLICATIONS

The present application is a national phase entry under 35 USC § 371 of International Application PCT/CN2022/137649, filed Dec. 8, 2022, which claims the benefit of and priority to Chinese Patent Application No. 202111572836.5, filed Dec. 21, 2021, and Chinese Patent Application No. 202211317991.7, filed Oct. 26, 2022.

### INCORPORATION BY REFERENCE

This application includes a sequence listing in computer readable form (a “xml” file) that is submitted herewith named P24SZ1NW00098US_sequencelist.xml created on Mar. 19, 2024, and 25,887 bytes in size. This sequence listing is incorporated by reference herein.

### TECHNICAL FIELD

The present disclosure belongs to the technical field of antibodies, in particular to human TIGIT antibody and use thereof.

### BACKGROUND

hTIGIT molecule is a costimulatory molecule with immunosuppression newly discovered in recent years. Researchers sequenced activated human T cells, and further studied some protein molecules with immunomodulator-like domains, and the result showed that both T cells and natural killer cells (NK) express hTIGIT. This molecule has an immunoglobulin-like domain, a transmembrane domain and an immunoreceptor tyrosine-based inhibitory motif (ITIM), so it is named T cell immunoglobulin and ITIM domain (hTIGIT). The hTIGIT gene is located on human chromosome 16 and encodes type I transmembrane protein consisting of 244 amino acids. Its sequence is conserved, and its homologous molecules have been found in many mammals. The human hTIGIT molecule has 88%, 67% and 58% homology with those of monkeys, dogs and mice, respectively.

Among the ligands of hTIGIT, it was discovered by bioinformatics analysis and in-vitro experiment that hTIGIT has the highest binding affinity to CD155, the second binding ability to CD113 and the lower binding affinity to CD112. Study has shown that after binding to the aforementioned ligands and being activated, hTIGIT can inhibit the activation of T cells by down-regulating three important molecules (TCRα, CD3ε and PLC-γ1) upstream of the TCR activation pathway, but also can up-regulate IL-2Rγ, CD25 and BCL-XL molecules which maintain the survival of T cells, preventing T cells from being eliminated. The NK cell is an important immune cell in the body, which plays an important role in anti-tumor, anti-virus and anti-intracellular bacterium aspects. Stanietsky et al. found that hTIGIT is not only expressed on the surface of T cells but also expressed on the surface of NK cells at a high level, and can transmit inhibitory signals through its intracellular ITIM motif to inhibit the killing function of NK cells, thus exerting a direct inhibiting effect on NK cells (FIG. 26).

Previous studies have shown that the expression of hTIGIT on the surface of T cells is increased in tumor progression, and hTIGIT is more expressed on the surface of NK cells in tumor than that on the surface of NK cells around the tumor. In the process of cancer-immunity cycle, hTIGIT can block the killing of tumor by immune cells through several steps. Firstly, hTIGIT can inhibit the effect of NK cells by preventing the initial death of tumor cells and releasing tumor antigens. hTIGIT can also inhibit the co-stimulatory capacity of dendritic cells, leading to the decrease of cancer antigen presentation and the increase of anti-inflammatory cytokines such as IL-10, and can induce PVR signal transduction in other cells such as tumor cells. In addition, hTIGIT can inhibit the effect of CD8+ T cells or deflect the polarization of CD4+ T cells. Finally, hTIGIT can directly inhibit the effect of CD8+ T cells, preventing the elimination of cancer cells (FIG. 26).

It is thus clear that hTIGIT can significantly inhibit immune cells from killing tumors, so inhibiting this molecule may become an effective anti-tumor target. A study has shown that a high-affinity blocking monoclonal antibody targeting mouse hTIGIT has been proved in an in-vitro experiment to have a strong blocking effect on hTIGIT receptor-ligand binding and enhance the function of NK cells, and it has been proved that an hTIGIT-based checkpoint immunotherapy can reverse the functional exhaustion of NK cells, enhance the anti-tumor immune response mediated by NK cells, effectively inhibit the growth of mouse tumors, and significantly prolong the survival time of tumor-bearing mice. In addition, this study has also proved that NK cells are a prerequisite for other checkpoint immunotherapies (e.g. anti-PD-L1) to achieve a curative effect. Mice successfully treated with this hTIGIT monoclonal antibody have a strong anti-tumor immunological memory for nearly a lifetime, and have strong resistance to the re-bearing of tumors without any treatment. To sum up, monoclonal antibodies targeting the inhibitory receptor hTIGIT have great potential for patent medicines and great development prospects for the treatment of solid tumors.

By August 2021, according to the information retrieved from the Biotech gate database, it can be seen that the clinical study on TIGIT targets has been actively promoted. The fastest progress in China is made by BGB-A1217 from BeiGene. Abroad, Tiragolumab from Roche (4.1D4, a reference antibody for the present application) has entered phase III clinical research. The above researches all take solid tumors as indications. The clinical trial research status on TIGIT targets by pharmaceutical companies are shown in Table 1.

TABLE 1
Summary of Main Information about Clinical Studies on Human TIGIT Targets (By August, 2021)

| Num- | | | Development | | For More |
|---|---|---|---|---|---|
| ber | Name | Company | Phase | Indication | Information |
| 1 | Tiragolumab (IgG1) | Roche | phase III | non-small cell lung cancer | NCT04513925 |
| | | | | and small cell lung cancer | |
| 2 | Vibostolimab, | Merck Sharp & | phase III | non-small cell lung cancer | NCT04738487 |
| | (MK-7684, IgG1) | Dohme | | | |
| 3 | Domvanalimab | Arcus Biosciences | phase III | non-small cell lung cancer | NCT04736173 |
| | (AB154) (IgG1) | | | | |
| 4 | BGB-A1217(IgG1) | BeiGene | phase III | advanced solid tumor | NCT04866017 |
| 5 | BMS-986207(IgG1) | BMS | phase II | advanced solid tumor | NCT05005273 |
| 6 | IBI-939(IgG1) | Innovent Biologics | phase I | advanced lung cancer | CTR20202565 |
| 7 | JS006 | TopAlliance | phase I | cancer | CTR20210449 |
| 8 | mAb-7 | Stainwei Biotech | preclinical | advanced solid tumor | |
| 9 | BAT6005 | BIO-THERA | preclinical | Cancer | CXSL2101040 |
| 10 | BAT6021 | BIO-THERA | preclinical | Cancer | CXSL2101052 |
| 11 | HB0030 | Huahai | preclinical | Cancer | CXSL2101098 |

### SUMMARY

The object in first aspect of the present disclosure is to provide a human TIGIT antibody or an antigen-binding fragment thereof.

The object in second aspect of the present disclosure is to provide a nucleic acid molecule encoding the human TIGIT antibody or the antigen-binding fragment thereof according to the first aspect of the present disclosure.

The object in third aspect of the present disclosure is to provide an expression cassette, a recombinant vector or a transgenic cell line containing the nucleic acid molecule according to the second aspect of the present disclosure.

The object in fourth aspect of the present disclosure is to provide an immunoconjugate.

The object in fifth aspect of the present disclosure is to provide a use of the human TIGIT antibody or the antigen-binding fragment thereof according to the first aspect of the present disclosure, the nucleic acid molecule according to the second aspect of the present disclosure, the expression cassette, recombinant vector or transgenic cell line according to the third aspect of the present disclosure and/or the immunoconjugate according to the fourth aspect of the present disclosure.

The object in sixth aspect of the present disclosure is to provide a product.

The object in seventh aspect of the present disclosure is to provide a pharmaceutical composition.

The object in eighth aspect of the present disclosure is to provide a method for treating tumors.

In order to achieve the above-mentioned objects, the present disclosure adopts the following technical solution:

In first aspect, the present disclosure provides a human TIGIT antibody, being 60H5 or 51C1, or an antigen-binding fragment thereof;
wherein 60H5 comprises a 60H5 heavy chain variable domain and a 60H5 light chain variable domain;
the 60H5 heavy chain variable domain comprises CDR1, CDR2, and CDR3;
the amino acid sequence of CDR1 of the 60H5 heavy chain variable domain comprises:
(a) GYTFTEYT (SEQ ID NO.: 2); or
(b) a sequence having 95%, 96%, 97%, 98% or 99% sequence identity with the amino acid sequence shown as SEQ ID NO.: 2;
the amino acid sequence of CDR2 of the 60H5 heavy chain variable domain comprises:
(a) INPNNGGT (SEQ ID NO.: 3); or
(b) a sequence having 95%, 96%, 97%, 98% or 99% sequence identity with the amino acid sequence shown as SEQ ID NO.: 3;
the amino acid sequence of CDR3 of the 60H5 heavy chain variable domain comprises:
(a) ARSGNWDYAMDY (SEQ ID NO.: 4); or
(b) a sequence having 95%, 96%, 97%, 98% or 99% sequence identity with the amino acid sequence shown as SEQ ID NO.: 4;
the 60H5 light chain variable domain comprises CDR1, CDR2, and CDR3;
the amino acid sequence of CDR1 of the 60H5 light chain variable domain comprises:
(a) QHVSTA (SEQ ID NO.: 7); or
(b) a sequence having 95%, 96%, 97%, 98% or 99% sequence identity with the amino acid sequence shown as SEQ ID NO.: 7;
the amino acid sequence of CDR2 of the 60H5 light chain variable domain comprises:
(a) SAS (SEQ ID NO.: 8); or
(b) a sequence having 95%, 96%, 97%, 98% or 99% sequence identity with the amino acid sequence shown as SEQ ID NO.: 8;
the amino acid sequence of CDR3 of the 60H5 light chain variable domain comprises:
(a) QQHYITPWT (SEQ ID NO.: 9); or
(b) a sequence having 95%, 96%, 97%, 98% or 99% sequence identity with the amino acid sequence shown as SEQ ID NO.: 9;
51C1 comprises a 51C1 heavy chain variable domain and a 51C1 light chain variable domain; the 51C1 heavy chain variable domain comprises CDR1, CDR2, and CDR3;
the amino acid sequence of CDR1 of the 51C1 heavy chain variable domain comprises:
(a) GYTFTEYF (SEQ ID NO.: 12); or
(b) a sequence having 95%, 96%, 97%, 98% or 99% sequence identity with the amino acid sequence shown as SEQ ID NO.: 12;
the amino acid sequence of CDR2 of the 51C1 heavy chain variable domain comprises:
(a) FYPGSGSI (SEQ ID NO.: 13); or
(b) a sequence having 95%, 96%, 97%, 98% or 99% sequence identity with the amino acid sequence shown as SEQ ID NO.: 13;
the amino acid sequence of CDR3 of the 51C1 heavy chain variable domain comprises:
(a) ARHEMRYGNYVLDY (SEQ ID NO.: 14); or
(b) a sequence having 95%, 96%, 97%, 98% or 99% sequence identity with the amino acid sequence shown as SEQ ID NO.: 14;
the 51C1 light chain variable domain comprises CDR1, CDR2, and CDR3;
the amino acid sequence of CDR1 of the 51C1 light chain variable domain comprises:
(a) TGAVTTRNY (SEQ ID NO.: 17); or
(b) a sequence having 95%, 96%, 97%, 98% or 99% sequence identity with the amino acid sequence shown as SEQ ID NO.: 17;
the amino acid sequence of CDR2 of the 51C1 light chain variable domain comprises:
(a) GTN (SEQ ID NO.: 18); or
(b) a sequence having 95%, 96%, 97%, 98% or 99% sequence identity with the amino acid sequence shown as SEQ ID NO.: 18;
the amino acid sequence of CDR3 of the 51C1 light chain variable domain comprises:
(a) GLWYSNHLV (SEQ ID NO.: 19); or
(b) a sequence having 95%, 96%, 97%, 98% or 99% sequence identity with the amino acid sequence shown as SEQ ID NO.: 19.

Preferably, the amino acid sequence of the 60H5 heavy chain variable domain comprises:
(a) EVQLQQSGPELVKPGASLKISCKTSGYTFTEYTMHWVKQSHGKSLEWIGGINPNN GGTKFKGKATLTVDKSSSTAYMELRSLTSEDSAVYYCARSGNWDYAMDYWGQGTSVTVSS (SEQ ID NO.: 1); or
(b) a sequence having 95%, 96%, 97%, 98% or 99% sequence identity with the amino acid sequence shown as SEQ ID NO.: 1; or
(c) QVQLVQSGAEVKKPGASVKVSCKTSGYTFTEYTMHWVRQAPGQRLEWIGGINPN NGGTSYNQKFQGRVTITVDTSASTAYMELSSLRSEDTAVYYCARSGNWDYAMDYWGQGT TVTVSS (SEQ ID NO.: 21); or
(d) a sequence having 95%, 96%, 97%, 98% or 99% sequence identity with the amino acid sequence shown as SEQ ID NO.: 21;
the amino acid sequence of the 60H5 light chain variable domain comprises:
(a) DIVMTQSHKFMSTSVGDRVSITCKASQHVSTAVVWYQQKPGQSPKLLIYSASYRYT GVDRFTGSGSGTDFTFTISSVQAEDLAVYYCQQHYITPWTFGGGTKLEIKRADA (SEQ ID NO.: 6); or
(b) a sequence having 95%, 96%, 97%, 98% or 99% sequence identity with the amino acid sequence shown as SEQ ID NO.: 6; or
(c) DIQMTQSPSSMSASVGDRVTITCKASQHVSTAVVWYQQKPGKAPKLLIYSASYRYT GVPDRFSGSGSGTDFTFTISSLQPEDIATYYCQQHYITPWTFGGGTKLEIKRTVA (SEQ ID NO.: 22); or
(d) a sequence having 95%, 96%, 97%, 98% or 99% sequence identity with the amino acid sequence shown as SEQ ID NO.: 22;
the amino acid sequence of the 51C1 heavy chain variable domain comprises:
(a) QVQLQQSGAELVKPGASVKLSCKASGYTFTEYFIHWIKQKSGQGLEWIGWFYPGS GSIKYNERFKDKATLTADKSSSTVYMELSRLTSEDSAVYFCARHEMRYGNYVLDYWGQGT TLTVSS (SEQ ID NO.: 11); or
(b) a sequence having 95%, 96%, 97%, 98% or 99% sequence identity with the amino acid sequence shown as SEQ ID NO.: 11; or
(c) QVQLVQSGAEVKKPGASVKVSCKASGYTFTEYFIHWVRQAPGQGLEWIGWFYPG SGSIKYNERFKDRVTLTADTSISTAYMELSRLRSDDTAVYYCARHEMRYGNYVLDYWGQGT TVTVSS (SEQ ID NO.: 23); or
(d) a sequence having 95%, 96%, 97%, 98% or 99% sequence identity with the amino acid sequence shown as SEQ ID NO.: 23;
the amino acid sequence of the 51C1 light chain variable domain comprises:
(a) QAVVTQESALTTSPGETVTLTCRSSTGAVTTRNYANWVQEKPDHLFTGLIGGTNNR VPGVPARFSGSLIGDKAALTITGAQTEDEAIYFCGLWYSNHLVFGGGTKLTVLGQPK (SEQ ID NO.: 16); or
(b) a sequence having 95%, 96%, 97%, 98% or 99% sequence identity with the amino acid sequence shown as SEQ ID NO.: 16; or
(c) QAVVTQEPSLTVSPGGTVTLTCRSSTGAVTTRNYANWVQQKPGQAPRGLIGGTNN RVPGVPARFSGSLLGGKAALTLSGAQPEDEAEYYCGLWYSNHLVFGGGTKLTVLGQPKA (SEQ ID NO.: 24); or
(d) a sequence having 95%, 96%, 97%, 98% or 99% sequence identity with the amino acid sequence shown as SEQ ID NO.: 24.

In second aspect, the present disclosure provides a nucleic acid molecule encoding the human TIGIT antibody or the antigen-binding fragment thereof according to the first aspect of the present disclosure.

In third aspect, the present disclosure provides an expression cassette, a recombinant vector or a transgenic cell line containing the nucleic acid molecule according to the second aspect of the present disclosure.

Preferably, the transgenic cell line does not contain animal and plant varieties.

In fourth aspect, the present disclosure provides an immunoconjugate, comprising the human TIGIT antibody or an antigen-binding fragment monoclonal antibody thereof or the antigen-binding fragment thereof according to the first aspect of the present disclosure and a conjugated portion.

The conjugated portion is at least one of a detectable marker, a drug, a toxin, a cytokine, an antibody, an antibody Fc fragment, an antibody scFv fragment, a radionuclide, an enzyme, a gold nanoparticle/nanorod, a magnetic nanoparticle, and a virus coat protein.

Preferably, the detectable marker is a fluorescent or luminescent marker.

Preferably, the radionuclide is at least one of a diagnostic isotope and a therapeutic isotope.

Preferably, the diagnostic isotope is at least one of Tc-99m, Ga-68, F-18, I-123, I-125, I-131, In-111, Ga-67, Cu-64, Zr-89, C-11, Lu-177, and Re-188.

Preferably, the therapeutic isotope is at least one of Lu-177, Y-90, Ac-225, As-211, Bi-212, Bi-213, Cs-137, Cr-51, Co-60, Dy-165, Er-169, Fm-255, Au-198, Ho-166, I-125, I-131, Ir-192, Fe-59, Pb-212, Mo-99, Pd-103, P-32, K-42, Re-186, Re-188, Sm-153, Ra223, Ru-106, Na24, Sr89, Tb-149, Th-227, Xe-133, Yb-169, and Yb-177.

Preferably, the drug is a cytotoxic drug.

Preferably, the cytotoxic drug is at least one of anti-tubulin drugs, DNA minor groove binding agents, DNA replication inhibitors, alkylating agents, antibiotics, folic acid antagonists, antimetabolites, chemosensitizers, topoisomerase inhibitors, and vincaalkaloids; and further, the cytotoxic drug is at least one of auristatins, camptothecins, duocarmycins, etoposides, maytansinoids and maytansinoids (e.g. DM1 and DM4), taxanes, benzodiazepines or benzodiazepinecontaining drugs (e.g. pyrrolo[1,4]benzodiazepines (PBDs), indolinobenzodiazepines and oxazolidinobenzodiazepines), and vincaalkaloids.

In fifth aspect, the present disclosure provides a use of the human TIGIT antibody or the antigen-binding fragment thereof according to the first aspect of the present disclosure, the nucleic acid molecule according to the second aspect of the present disclosure, the expression cassette, recombinant vector or transgenic cell line according to the third aspect of the present disclosure and the immunoconjugate according to the fourth aspect of the present disclosure.

The use of any one of (1) to (4) in any one of (5) and (6);
(1) the human TIGIT antibody or the antigen-binding fragment thereof according to the first aspect of the present disclosure;
(2) the nucleic acid molecule according to the second aspect of the present disclosure;
(3) the expression cassette, recombinant vector or transgenic cell line according to the third aspect of the present disclosure;
(4) the immunoconjugate according to the fourth aspect of the present disclosure;
(5) the preparation of a product for assaying TIGIT;
(6) the preparation of a drug for treating a tumor.

Preferably, the TIGIT mentioned in (5) is at least one of human TIGIT and monkey TIGIT; and further, TIGIT is human TIGIT.

Preferably, the product mentioned in (5) is at least one of a reagent, an assay plate, and a kit.

Preferably, the tumor mentioned in (6) comprises hematological neoplasms, solid tumors, non-small cell lung cancer, small cell lung cancer, colorectal cancer, melanoma, breast cancer, esophageal cancer, gastric tumors, bladder cancer, endometrial cancer, head and neck cancer, and renal cancer; and further, the tumor is at least one of colorectal cancer and breast cancer.

In sixth aspect, the present disclosure provides a product, comprising at least one of the human TIGIT antibody or the antigen-binding fragment thereof according to the first aspect of the present disclosure and the immunoconjugate according to the fourth aspect of the present disclosure; and the product is at least one of a reagent, an assay plate, and a kit.

Preferably, the product is used for assaying TIGIT.

Preferably, TIGIT is at least one of human TIGIT and monkey TIGIT; and further, TIGIT is human TIGIT.

In seventh aspect, the present disclosure provides a pharmaceutical composition, comprising at least one of (1) to (4);

(1) the human TIGIT antibody or the antigen-binding fragment thereof according to the first aspect of the present disclosure;

(2) the nucleic acid molecule according to the second aspect of the present disclosure;

(3) the expression cassette, recombinant vector or transgenic cell line according to the third aspect of the present disclosure;

(4) the immunoconjugate according to the fourth aspect of the present disclosure.

Preferably, the pharmaceutical composition further comprises a pharmaceutically acceptable carrier.

Preferably, the pharmaceutical composition is used for treating a tumor.

Preferably, the tumor comprises hematological neoplasms, solid tumors, non-small cell lung cancer, small cell lung cancer, colorectal cancer, melanoma, breast cancer, esophageal cancer, gastric tumors, bladder cancer, endometrial cancer, head and neck cancer, and renal cancer; and further, the tumor is at least one of colorectal cancer and breast cancer.

Preferably, the conjugated portion of the immunoconjugate is a drug, a toxin, and/or a therapeutic isotope.

In eighth aspect, the present disclosure provides a method for treating tumors, comprising the step of administering an effective dose of the human TIGIT antibody or the antigen-binding fragment thereof according to the first aspect of the present disclosure, the nucleic acid molecule according to the second aspect of the present disclosure, the expression cassette, recombinant vector or transgenic cell line according to the third aspect of the present disclosure or the immunoconjugate according to the fourth aspect of the present disclosure to a patient.

The beneficial effects of the present disclosure are as follows:

The present disclosure provides a human TIGIT antibody or the antigen-binding fragment thereof, which has strong binding capability with hTIGIT and can induce T cells of B-hTIGIT transgenic mice, Jurkat cells and human PBMC cells to secrete cytokines (IL-2 and/or IFN-γ). It has remarkable ADCC effect and CDC effect and can significantly reduce the tumor volume/weight of colon cancer cell line CT26 tumor-bearing mice and breast cancer cell line 4T1 tumor-bearing mice without affecting the body weights of the mice (i.e. no side effects), prolong the survival time of mice, and has good pharmacokinetic characteristics, which can be used for assaying hTIGIT and treating tumors.

### BRIEF DESCRIPTION OF DRAWINGS

FIG. 1 is a result graph of the binding activities of hTIGIT mouse-derived antibodies (m60H5 and m51C1) with an antigen hTIGIT-his protein in Example 3.

FIG. 2 is a result graph of the binding activities of the hTIGIT mouse-derived antibodies (m60H5 and m51C1) with hTIGIT on a 293F-hTIGIT-short #1B1 cell line in Example 3.

FIG. 3 is a result graph of the competitive activities of the hTIGIT mouse-derived antibodies (m60H5 and m51C1) in competing with PVR for binding with hTIGIT on the surface of the 293F-hTIGIT-short cells in Example 4.

FIGS. 4A-4B are result graphs of the binding activities of the hTIGIT mouse-derived antibodies (m60H5 and m51C1) with monkey- and mouse-derived TIGIT proteins in Example 5, where FIG. 4A is the result graph of the binding activities of the hTIGIT mouse-derived antibodies (m60H5 and m51C1) with the monkey-derived TIGIT protein, and FIG. 4B is the result graph of the binding activities of the hTIGIT mouse-derived antibodies (m60H5 and m51C1) with the mouse-derived TIGIT protein.

FIGS. 5A-5B are result graphs of the binding activities of hTIGIT humanized antibodies (h60H5 and h51C1) with hTIGIT in Example 6, where FIG. 5A is the result graph of the binding activities of the hTIGIT humanized antibodies (h60H5 and h51C1) with hTIGIT assayed by ELISA, and FIG. 5B is the result graph of the binding activities of the hTIGIT humanized antibodies (h60H5 and h51C1) with an hTIGIT protein expressed on the surface of cells assayed by flow cytometry (FCM).

FIG. 6 is a result graph of the competitive activities of the hTIGIT humanized antibodies (h60H5 and h51C1) in competing with PVR for binding with hTIGIT on the surface of the 293F-hTIGIT-short cells in Example 7.

FIG. 7 is an influence diagram of the level of secretion of mIL-2 by T cells of B-hTIGIT transgenic mice activated by the hTIGIT mouse-derived antibodies (m60H5 and m51C1) in Example 8.

FIG. 8 is an influence diagram of the level of secretion of mIFN-γ by T cells of B-hTIGIT transgenic mice activated by the hTIGIT mouse-derived antibodies (m60H5 and m51C1) in Example 8.

FIG. 9 is an influence diagram of the level of secretion of mIL-2 by T cells of B-hTIGIT transgenic mice activated by the hTIGIT humanized antibodies (h60H5 and h51C1) in Example 9.

FIG. 10 is an influence diagram of the level of secretion of mIFN-γ by T cells of B-hTIGIT transgenic mice activated by the hTIGIT humanized antibodies (h60H5 and h51C1) in Example 9.

FIG. 11 is an influence diagram of the level of secretion of human IL-2 by Jurkat cells activated by the hTIGIT mouse-derived antibodies (m60H5 and m51C1) in Example 10.

FIG. 12 is an influence diagram of the level of secretion of human IL-2 by Jurkat cells activated by the hTIGIT humanized antibodies (h60H5 and h51C1) in Example 11.

FIG. 13 is an influence diagram of the level of secretion of human IL-2 by human PBMC cells activated by the hTIGIT mouse-derived antibodies (m60H5 and m51C1) in Example 12.

FIG. 14 is an influence diagram of the level of secretion of human IFN-γ by human PBMC cells activated by the hTIGIT mouse-derived antibodies (m60H5 and m51C1) in Example.

FIG. 15 is an influence diagram of the level of secretion of human IFN-γ by human PBMC cells activated by the hTIGIT humanized antibodies (h60H5 and h51C1) in Example 13.

FIG. 16 is an influence diagram of the level of secretion of human IL-2 by human PBMC cells activated by the hTIGIT humanized antibodies (h60H5 and h51C1) in Example 13.

FIG. 17 is a graph of the competition between the hTIGIT mouse-derived antibodies (m60H5 and m51C1) and 4.1D3 for hTIGIT epitope binding in Example 14.

FIGS. 18A-18B are ADCC effect graphs of the hTIGIT humanized antibodies (h60H5 and h51C1) in Example 15, where FIG. 18A is the ADCC effect graph of the hTIGIT humanized antibody h60H5; and FIG. 18B is the ADCC effect graph of the hTIGIT humanized antibody h51C1.

FIGS. 19A-19B are CDC effect graphs of the hTIGIT humanized antibodies (h60H5 and h51C1) in Example 16, where FIG. 19A is the CDC effect graph of the hTIGIT humanized antibody h60H5; and FIG. 19B is the CDC effect graph of the hTIGIT humanized antibody h51C1.

FIGS. 20A-20B are influence diagrams of the hTIGIT humanized antibodies (h60H5 and h51C1) on colon cancer cell line CT26 tumor-bearing mice in Example 17, where FIG. 20A is the influence diagram of the hTIGIT humanized antibodies (h60H5 and h51C1) on the tumor volume of the colon cancer cell line CT26 tumor-bearing mice, and FIG. 20B is the influence diagram of the hTIGIT humanized antibodies (h60H5 and h51C1) on the body weights of the colon cancer cell line CT26 tumor-bearing mice.

FIG. 21 is a survival graph of the colon cancer cell line CT26 tumor-bearing mice treated by the hTIGIT humanized antibodies (h60H5 and h51C1) in Example 17.

FIGS. 22A-22B are result graphs of a tumor re-challenge experiment on the cured tumor-bearing mice in Example 18, where FIG. 22A is the efficacy (tumor size)-drug concentration graph after the re-challenge of BALB/C-huTIGIT colon cancer cells CT 26 in humanized mice, and FIG. 22B is the graph of body weight change after the humanized mice are re-inoculated with CT 26 for re-challenge.

FIGS. 23A-23C are influence diagrams of the hTIGIT humanized antibodies (h60H5 and h51C1) on breast cancer cell 4T1 tumor-bearing B-huTIGIT mice in Example 19, where FIG. 23A is the influence diagram of the hTIGIT humanized antibodies (h60H5 and h51C1) on the tumor volume of the breast cancer cell 4T1 tumor-bearing B-huTIGIT mice, FIG. 23B is the influence diagram of the hTIGIT humanized antibodies (h60H5 and h51C1) on the tumor weight of the breast cancer cell 4T1 tumor-bearing B-huTIGIT mice, and FIG. 23C is the influence diagram of the hTIGIT humanized antibodies (h60H5 and h51C1) on the body weights of the breast cancer cell 4T1 tumor-bearing B-huTIGIT mice.

FIG. 24 is a survival graph of the breast cancer cell 4T1 tumor-bearing B-huTIGIT mice treated by the hTIGIT humanized antibodies (h60H5 and h51C1) in Example 19.

FIG. 25 is a pharmacokinetic result graph of the hTIGIT humanized antibodies (h60H5 and h51C1) in Example 20.

FIG. 26 is a schematic diagram of the signal transduction mechanism of the TIGIT protein in T cells and NK cells.

### DETAILED DESCRIPTION

The content of the present disclosure will be further illustrated in detail through specific examples.

It should be understood that these examples are merely intended to illustrate the present disclosure rather than limit the scope of the present disclosure.

The experimental methods without specific conditions in the following examples are generally in accordance with conventional conditions or conditions recommended by the manufacturers. Unless otherwise specified, the materials, reagents and so on used in the examples are commercially available reagents and materials.

### Example 1: Preparation of Human TIGIT Mouse-derived Antibodies

The hybridoma technique was used to produce human TIGIT mouse-derived monoclonal antibodies, as follows:

Balb/c mice were immunized with hTIGIT (NP_776160.2) as an antigen. The immunized mice were immunized with a purified antigen and complete Freund's adjuvant for three times, and the immune response was assayed after bloodletting through the caudal vein. Serum was screened by ELISA and flow cytometry to acquire the mice with human TIGIT immunoglobulin. Splenocytes were gotten from the mice with the highest anti-TIGIT immunoglobulin and fused with mouse myeloma cells SP2/0 (ATCC number: CRL-1581). The fused hybridoma cells were screened for antibodies, obtaining the human TIGIT mouse-derived antibodies: m60H5 and m51C1. The human TIGIT mouse-derived antibodies (m60H5 and m51C1) were sequenced.

The amino acid sequence of the heavy chain variable domain of m60H5 is

(SEQ ID NO.: 1
EVQLQQSGPELVKPGASLKISCKTSGYTFTEYTMHWVKQSHGKSLEWIG
GINPNNGGTKFKGKATLTVDKSSSTAYMELRSLTSEDSAVYYCARSGNW
DYAMDYWGQGTSVTVSS,

where the underlined portions are CDR1 (SEQ ID NO.: 2), CDR2 (SEQ ID NO.: 3) and CDR3 (SEQ ID NO.: 4) in sequence), and the nucleotide sequence encoding the heavy chain variable domain of m60H5 with the amino acid sequence shown as SEQ ID NO.: 1 is GAGGTCCAGCTGCAACAATCTGGACCTGAGCTGGTGAAGCCTGGGGCTTCACTGAAGA TATCCTGCAAGACTTCTGGATACACATTCACTGAATACACCATGCACTGGGTGAAGCAGA GCCATGGAAAGAGCCTTGAGTGGATTGGAGGTATTAATCCTAACAATGGTGGTACTAAGT TCAAGGGCAAGGCCACATTGACTGTAGACAAGTCCTCCAGCACAGCCTACATGGAGCTC CGCAGCCTGACATCTGAGGATTCTGCAGTCTATTACTGTGCAAGATCGGGGAACTGGGA CTATGCTATGGACTACTGGGGTCAAGGAACCTCAGTCACCGTCTCCTCA (SEQ ID NO.: 5). The amino acid sequence of the light chain variable domain of m60H5 is

(SEQ ID NO.: 6
DIVMTQSHKFMSTSVGDRVSITCKASQHVSTAVVWYQQKPGQSPKLLIY
SASYRYTGVDRFTGSGSGTDFTFTISSVQAEDLAVYYCQQHYITPWTFG
GGTKLEIKRADA,

where the underlined portions are CDR1 (SEQ ID NO.: 7), CDR2 (SEQ ID NO.: 8) and CDR3 (SEQ ID NO.: 9) in sequence), and the nucleotide sequence encoding the light chain variable domain of m60H5 with the amino acid sequence shown as SEQ ID NO.: 6 is GACATTGTGATGACCCAGTCTCACAAATTCATGTCCACATCAGTAGGAGACAGGGTCAG CATCACCTGCAAGGCCAGTCAACATGTGAGTACTGCTGTAGTCTGGTATCAACAGAAAC CAGGACAATCTCCTAAACTACTGATTTACTCGGCATCGTACCGGTACACTGGAGTCCCTG ATCGGTTCACTGGCAGTGGATCTGGGACGGATTTCACTTTCACCATCAGCAGTGTGCAG GCTGAGGACCTGGCAGTTTATTACTGTCAGCAACATTATATTACTCCGTGGACGTTCGGT GGAGGCACCAAGCTGGAAATCAAACGGGCTGAT (SEQ ID NO.: 10).

The amino acid sequence of the heavy chain variable domain of m51C1 is

(SEQ ID NO.: 11
QVQLQQSGAELVKPGASVKLSCKASGYTFTEYFIHWIKQKSGQGLEWIG
WFYPGSGSIKYNERFKDKATLTADKSSSTVYMELSRLTSEDSAVYFCAR
HEMRYGNYVLDYWGQGTTLTVSS,

where the underlined portions are CDR1 (SEQ ID NO.: 12), CDR2 (SEQ ID NO.: 1 3) and CDR3 (SEQ ID NO.: 14) in sequence), and the nucleotide sequence encoding the heavy chain variable domain of m51C1 with the amino acid sequence shown as SEQ ID NO.: 11 is CAGGTCCAGCTGCAGCAGTCAGGAGCTGAGCTGGTGAAACCCGGGGCATCAGTG AAGCTGTCCTGTAAGGCTTCTGGCTACACCTTCACTGAGTATTTTATACACTGGATAAAG CAGAAGTCTGGACAGGGTCTTGAGTGGATTGGGTGGTTTTACCCTGGAAGTGGTAGTAT AAAGTACAATGAGAGATTCAAGGACAAGGCCACATTGACTGCGGACAAATCCTCCAGC ACAGTCTATATGGAGCTTAGTAGATTGACATCTGAAGACTCTGCGGTCTATTTCTGTGCA AGACACGAGATGAGGTATGGTAACTACGTCCTTGACTACTGGGGCCAAGGCACCACTCT CACAGTCTCCTCA (SEQ ID NO.: 15). The amino acid sequence of the light chain variable domain of m51C1 is

(SEQ ID NO.: 16
QAVVTQESALTTSPGETVTLTCRSSTGAVTTRNYANWVQEKPDHL
FTGLIGGTNNRVPGVPARFSGSLIGDKAALTITGAQTEDEAIYFCGLWY
SNHLVFGGGTKLTVLGQPK,

where the underlined portions are CDR1 (SEQ ID NO.: 17), CDR2 (SEQ ID NO.: 18) and CDR3 (SEQ ID NO.: 19) in sequence), and the nucleotide sequence encoding the light chain variable domain of m51C1 with the amino acid sequence shown as SEQ ID NO.: 16 is CAGGCTGTTGTGACTCAGGAATCTGCACTCACCACATCACCT GGTGAAACAGTCACACTCACTTGTCGCTCAAGTACTGGGGCTGTTACAACTAGAAACTA TGCCAACTGGGTCCAAGAAAAACCAGATCATTTATTCACAGGTCTAATAGGTGGTACCA ACAACCGAGTTCCAGGTGTTCCTGCCAGATTCTCAGGCTCCCTGATTGGAGACAAGGCT GCCCTCACCATCACAGGGGCACAGACTGAGGATGAGGCAATTTATTTCTGTGGTCTATG GTACAGCAACCATTTGGTGTTCGGTGGAGGAACCAAACTGACTGTCCTAGGCCAGCCCA AG (SEQ ID NO.: 20).

### Example 2: Preparation of Human TIGIT Humanized Antibodies

Referring to the sequences of the light chain variable domains and heavy chain variable domains of the m60H5 and m51C1 antibodies, humanized templates that best matched their non-CDR regions were chosen. The humanized antibodies (h60H5 and h51C1) were obtained by transplanting the CDR regions of the mouse-derived antibodies onto the chosen humanized templates to replace the CDR regions of the humanized templates. Then, based on the three-dimensional structure of the mouse-derived antibodies, the embedded residues, the residues in direct interaction with the CDR regions and the residues having important influence on the conformations of VL and VH were reversely mutated to obtain the humanized antibodies. The sequence of the heavy chain variable domain of the humanized antibody h60H is: QVQLVQSGAEVKKPGASVKVSCKTSGYTFTEYTMHWVRQAPGQRLEWIGGINPNNG GTSYNQKFQGRVTITVDTSASTAYMELSSLRSEDTAVYYCARSGNWDYAMDYWGQGTTVT VSS (SEQ ID NO: 21), and the sequence of the light chain variable domain of the humanized antibody h60H5 is: DIQMTQSPSSMSASVGDRVTITCKASQHVSTAVVWYQQKPGKAP KLLIYSASYRYTGVPDRFSGSGSGTDFTFTISSLQPEDIATYYCQQHYITPWTFGGGTKLEIK RTVA (SEQ ID NO: 22). The sequence of the heavy chain variable domain of the humanized antibody h51C1 is: QVQLVQSGAEVKKPGASVKVSCKASGYTFTEYFIHWVRQAPGQG LEWIGWFYPGSGSIKYNERFKDRVTLTADTSISTAYMELSRLRSDDTAVYYCARHEMRYGN YVLDYWGQGTTVTVSS (SEQ ID NO: 23), and the sequence of the light chain variable domain of the humanized antibody h51C1 is: QAVVTQEPSLTVSPGGTVTLTCRSSTGAVTT RNYANWVQQKPGQAPRGLIGGTNNRVPGVPARFSGSLLGGKAALTLSGAQPEDEAEYYCG LWYSNHLVFGGGTKLTVLGQPKA (SEQ ID NO: 24).

### Example 3: Binding Activities of hTIGIT Mouse-Derived Antibodies with hTIGIT

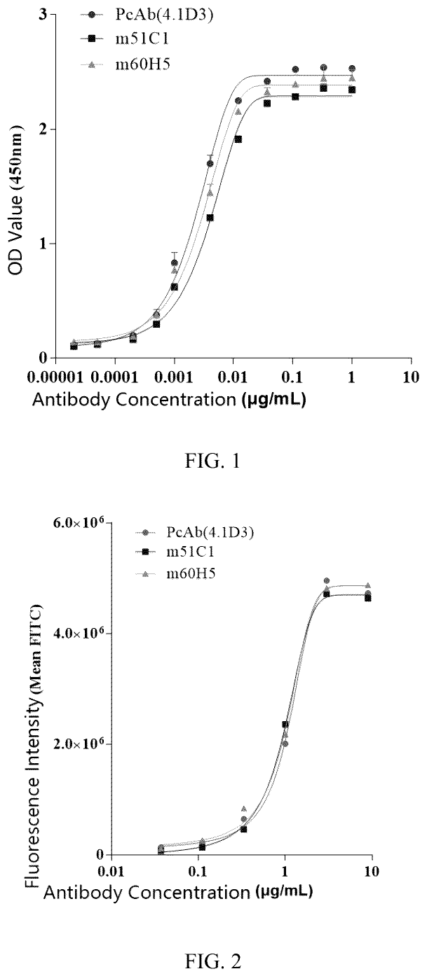

The binding activities of the hTIGIT mouse-derived antibodies (m60H5 and m51C1) and a positive control PcAb (4.1D3, purchased from Sanyou Bio, WJ20201031) with an hTIGIT-His protein were assayed by ELISA. The hTIGIT-his protein was diluted to 0.5 μg/mL with CBS buffer, coated in an ELISA plate in 50 μL/well, and left at 4° C. overnight. The plate was washed with PBST buffer for three times, 200 μL/well, and patted dry. 1% of BSA (dissolved in PBS) was then added, 200 μL/well, and the plate was incubated at 37° C. for 2 hours. After incubation, the plate was washed with PBST for three times, 200 μL/well, and patted dry. The positive control PcAb (4.1D3) and the hTIGIT mouse-derived antibodies (m60H5 and m51C1) were diluted to 1 μg/mL with PBST, gradient dilution was performed from column 1 to column 11 (eleven groups in total) in a dilution plate according to a 3-fold dilution method, and the 12th group was added with 1×PBST as a blank control group. 100 μL was sucked with a multi-channel pipette, transferred into the ELISA plate, and incubated at 37° C. for 1 hour. After incubation, the plate was washed with PBST for three times, 200 μL/well, and patted dry. An anti-mouse IgG antibody labeled by HRP was diluted with 1% of BSA (dissolved in PBST) (diluted at 1:5000), 100 μL/well. Incubation was performed at 37° C. for 1 hour. After incubation, the plate was washed with PBST for three times, 200 μL/well, and patted dry. 50 μL of TMB chromogen solution was added into each well for chromogenic reaction, and the reaction time was 5 minutes in the dark. After 5 minutes of incubation, hydrochloric acid stop solution was directly added to stop the reaction in 50 μL/well. Absorbances at 450 nm (OD450) were detected by the light absorption mode of a microplate reader. The ELISA assay results shown in FIG. 1: the hTIGIT mouse-derived antibodies (m60H5 and m51C1) and the positive control PcAb (4.1D3) have strong binding capability with the hTIGIT-His protein, and the binding activities of the obtained mouse-derived antibodies (m60H5 (EC50=0.00277 μg/mL) and m51C1 (EC50=0.00396 μg/mL)) are similar to that of the positive control (4.1D3 (EC50=0.00255 μg/mL)).

293F-hTIGIT-short #1B1 cells were constructed as follows: the gene sequences of the extracellular domain and transmembrane domain of TIGIT (synthesized by General Biol, Anhui, China, Gene ID: 201633) were synthesized and then inserted the gene sequences of the extracellular domain and transmembrane domain of TIGIT between EcoRI-BamHI of pLVX-Puro plasmid. The recombinant plasmid was then transfected into 293F cells (purchased from ATCC), and a stable cell line capable of stably expressing the TIGIT protein was formed under the screening effect of cell medium containing 2.5 μg/mL of puromycin. The cell line was then monoclonalized by a limiting dilution method. After screening and identification, it was determined that clone #1B1 was an extracellular domain-based monoclonal cell line capable of stably expressing TIGIT.

The 293F-hTIGIT-short #1B1 cell line was digested, terminated, centrifuged, resuspended and counted, and the cell density was adjusted to 4×10⁶ cells/mL. According to 50 μL/tube, 20×10⁴ cells were added into 1.5 mL EP tubes. The prepared hTIGIT mouse-derived antibodies (m60H5 and m51C1) or positive control PcAb (4.1D3) were added. The hTIGIT mouse-derived antibodies and PcAb (4.1D3) were diluted to an initial concentration of 12 μg/mL, seven concentrations were obtained by gradient dilution in 1:3, they were added into the corresponding tubes in 150 μL/tube. The blank group was added with 150 μL of PBS as a blank control. Gentle blowing and mixing were performed, and incubation was performed on ice for 1 hour. After incubation, 500 μL of PBS was added for centrifugal washing once, centrifugation was performed at 2000 rpm for 5 minutes, and the supernatants were discarded. A fluorescent secondary antibody was added (Alexa® 488 fluorescently labeled anti-mouse IgG antibody was diluted with PBS (diluted at 1:500) and added to the corresponding tubes in 200 μL/tube), gentle blowing and mixing were performed, and incubation was performed on ice in the dark for 30 minutes. After 30 minutes of incubation, signal detection was performed with a flow cytometer. The binding activities of the hTIGIT mouse-derived antibodies or the positive control PcAb (4.1D3) were analyzed to obtain EC50 values. Flow cytometry (FCM) was used to assay the binding activities of the hTIGIT mouse-derived antibodies and the positive control PcAb (4.1D3) with hTIGIT on the surface of the 293F-hTIGIT-short cells. The FCM assay results shown in FIG. 2: the hTIGIT mouse-derived antibodies (m60H5 (EC50=0.631 μg/mL) and m51C1 (EC50=0.811 μg/mL)) and the positive control PcAb (4.1D3, EC50=1.11 μg/mL) have strong binding capability with hTIGIT on the cell surface, and the binding activities of the mouse-derived antibodies (m60H5 and m51C1) are equivalent to that of the positive control.

### Example 4: Competitive Binding Activities of hTIGIT Mouse-Derived Antibodies

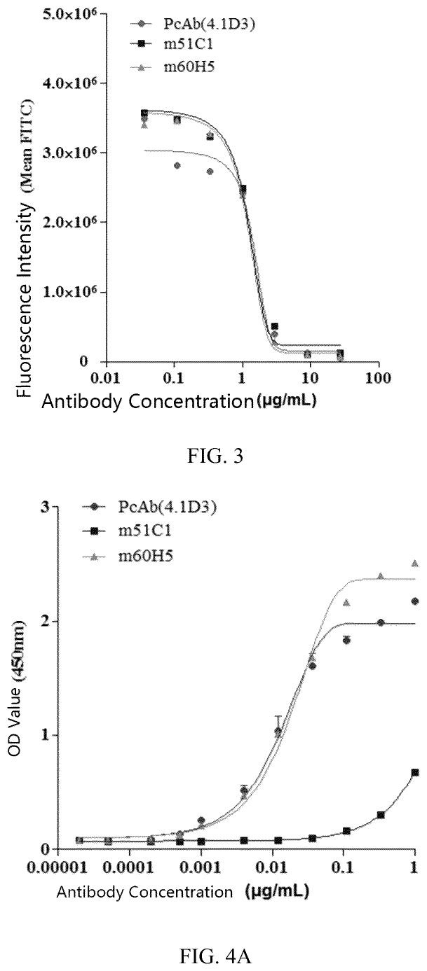

The 293F-hTIGIT-short #1B1 cell line was digested, terminated, centrifuged, resuspended and counted, and the cell density was adjusted to 4×10⁶ cells/mL. According to 50 μL/tube, 20×10⁴ cells were added into 1.5 mL EP tubes. The hTIGIT mouse-derived antibodies (m60H5 and m51C1) or PcAb (4.1D3) were diluted to an initial concentration of 108 μg/mL, seven concentrations were obtained by gradient dilution in 1:3, and they were added into the corresponding tubes in 50 μL/tube. The blank group, the PVR-hFc group (added with hTIGIT mouse-derived antibodies (m60H5 and m51C1)) and the PVR-mFc group (added with PcAb (4.1D3)) were added with 50 μL of PBS respectively, gentle blowing and mixing were performed, and incubation was performed on ice for 15 minutes first. After 15 minutes, the prepared PVR-hFc protein (6 μg/mL, sequence as SEQ ID NO.: 25) was added to the PVR-hFc group and the prepared PVR-mFc protein (6 μg/mL, sequence as SEQ ID NO.: 26) was added to the PVR-mFc group, 100 μL/tube. 100 μL of PBS was added into the blank group. Gentle blowing and mixing were performed, and incubation was then performed on ice for 1 hour. After incubation, 500 μL of PBS was added for centrifugal washing once, centrifugation was performed at 2000 rpm for 5 minutes, and the supernatants were discarded. Alexa® 488 fluorescently labeled anti-mouse or anti-human IgG antibody was diluted with PBS (diluted at 1:500) and added to the corresponding tubes in 200 μL/tube, gentle blowing and mixing were performed, and incubation was performed on ice in the dark for 30 minutes. After 30 minutes of incubation, signal detection was performed with a flow cytometer. It can be seen from the antagonistic activity results of the mouse-derived antibodies (m60H5 and m51C1) that the antagonistic activities of both antibodies are similar to that of 4.1D3 (FIG. 3). The competitive activity of each antibody is concentration-dependent. The IC50 values of m60H5, m51C1 and 4.1D3 are 1.54 μg/mL, 1.35 μg/mL and 1.27 μg/mL respectively.

### Example 5: Cross-binding Activities of hTIGIT Mouse-Derived Antibodies with Monkey- and Mouse-Derived TIGIT Proteins

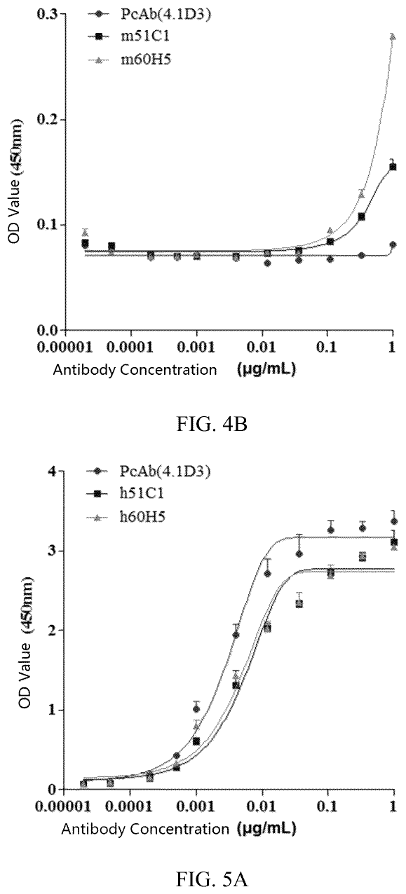

The binding activities of the hTIGIT mouse-derived antibodies (m60H5 and m51C1) and the positive control PcAb (4.1D3) with a monkey-derived TIGIT protein (accession number: XP_016797180.2) or a mouse-derived TIGIT protein (accession number: NP_001139797.1) were analyzed by detection method of indirect ELISA. The method was the same as that example 3. From the homology of the TIGIT protein sequences of human, monkey and mouse, the TIGIT protein of human has high homology with that of monkey, but has low homology with that of mouse. The results shown in FIG. 4A-B: the hTIGIT mouse-derived antibodies (m60H5 and m51C1) and the positive control PcAb (4.1D3) maintain the binding activities with the monkey-derived TIGIT protein. In particular, m60H5 and the positive control PcAb (4.1D3) have high binding activities with the monkey-derived TIGIT protein, while m51C1 has weak affinity with the monkey-derived TIGIT protein. The binding capability of the hTIGIT mouse-derived antibodies (m60H5 and m51C1) and the positive control PcAb (4.1D3) with the mouse-derived TIGIT protein is very weak, which can be regarded as no affinity.

### Example 6: Binding Activities of hTIGIT Humanized Antibodies with hTIGIT

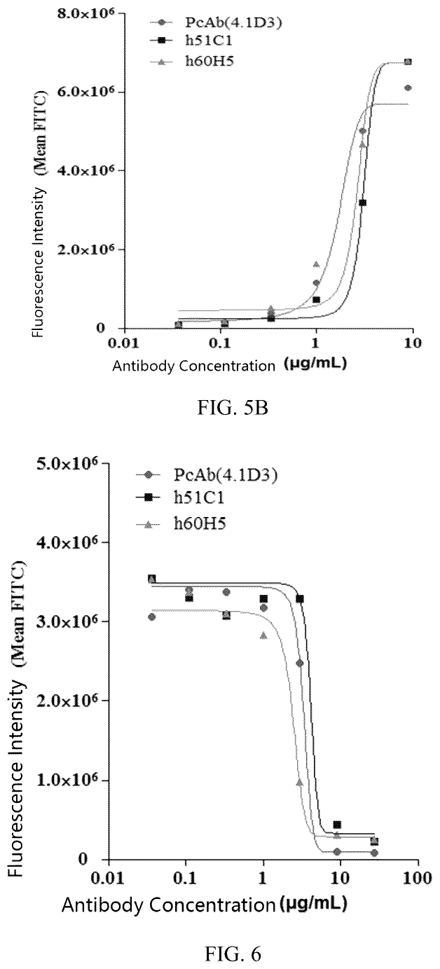

The binding activities of the hTIGIT humanized antibodies (h60H5 and h51C1) and the positive control PcAb (4.1D3) with the hTIGIT proteins were assayed by ELISA and flow cytometry respectively. The method was the same as that in example 3. The ELISA results shown in FIG. 5A: the hTIGIT humanized antibodies (h60H5 (EC50=0.006 μg/mL), h51C1 (EC50=0.007 μg/mL)) and the positive control PcAb (4.1D3, EC50=0.003 μg/mL) all have strong binding activities with the hTIGIT-His protein. The FCM assay results shown in FIG. 5B: the hTIGIT humanized antibodies (h60H5 (EC50=1.57 μg/mL), h51C1 (EC50=2.98 μg/mL)) and the positive control PcAb (4.1D3, EC50=1.05 μg/mL) have strong binding capability with hTIGIT on the cell surface, and the binding activities of the three are equivalent.

### Example 7: Competitive Binding Activities of hTIGIT Humanized Antibodies

The capabilities of the hTIGIT humanized antibodies (h60H5 and h51C1) and the positive control PcAb (4.1D3) in competing with hTIGIT competing poliovirus receptor (PVR) for binding with TIGIT were assayed by flow cytometry (the method was the same as that in example 4). The results shown in FIG. 6: the hTIGIT humanized antibodies (h60H5 (IC50=2.00 μg/mL), h51C1 (IC50=5.21 μg/mL)) and the positive control PcAb (4.1D3, IC50=3.77 μg/mL) all have strong competitive activities against PVR, and in particular, the competitive activity of h60H5 is better than those of h51C1 and the positive control PcAb (4.1D3).

### Example 8: Secretion of Cytokines by Inducing T Cells of B-hTIGIT Transgenic Mice with hTIGIT Mouse-Derived Antibodies

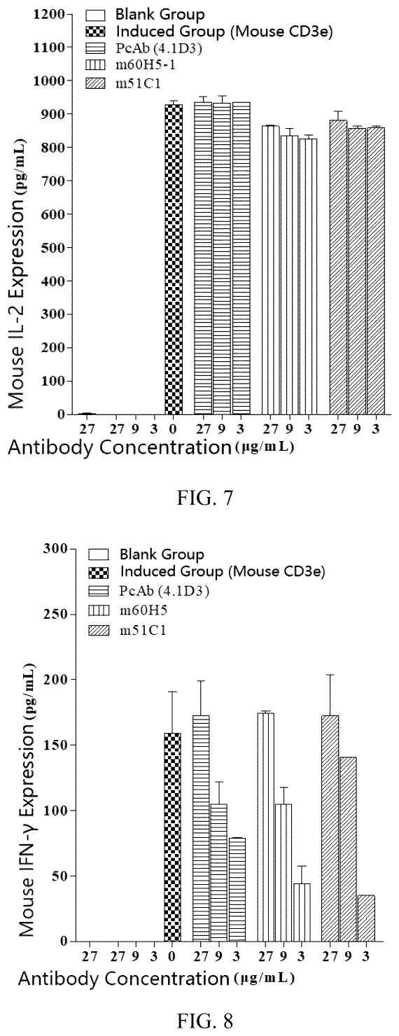

The B-hTIGIT transgenic mice (purchased from GemPharmatech, Nanjing, China) were killed by eyeball enucleation, bloodletting and cervical dislocation, the spleens of the mice were then aseptically taken out, and the mucous membranes and other tissues on the surface of the spleens were removed. The spleens were placed on a 70 μm sieve, wetted with serum-free DMEM, cut into pieces, and ground with the core of a 2.5 mL sterile syringe, and the sieve was then rinsed with 3 mL of serum-free DMEM until the spleen tissue was fully ground. The cell suspension was then transferred into a 40 μm sieve with a 10 mL pipette for re-filtration. The filtered cell suspension was centrifuged at 1000 rpm for 5 minutes, the supernatant was sucked off. 3 mL of PBS was then added for washing once. Centrifugation was performed at 1000 rpm for 5 minutes, the supernatant was sucked off. 1 mL of DMEM complete medium (containing 10% of FBS) was added to resuspend the cells. The cell suspension was transferred into a T75 flask, DMEM complete medium was replenished to 15 mL, and the cells were put into a cell incubator at 37° C. and 5% of CO₂ for continuous culture. The Anti-mouse CD3e protein was coated in a concentration of 10 μg/mL (diluting the Anti-mouse CD3e protein to 10 μg/mL with sterile CBS, adding the diluted protein into a 96-well plate in 50 μL/well, adding an adhesive film for sealing, and performing incubation overnight at 4° C.). The supernatant was discarded from the 96-well plate with the Anti-mouse CD3e protein coated overnight, patted dry and then washed with sterile PBS once. The supernatant was discarded, and patted dry. The splenocytes of the mice were resuspended by centrifugation and counted. The cell density was adjusted to 2×10⁶ cells/mL, and the cells were added into the 96-well plate in 0.1×10⁶ cells/well and 50 μL/well. The corresponding antibody (the hTIGIT mouse-derived antibodies (m60H5 and m51C1) and PcAb (4.1D3)) concentrations were prepared: the antibodies were diluted from an initial concentration of 40.5 μg/mL (1.5×), three concentrations were obtained by gradient dilution in 1:3, and the diluents were DMEM complete medium (containing 10% of FBS). The antibody group and the induced group (added with an equal volume of PBS) were added into the cell wells in 100 μL/well, the DMEM complete medium was added into the blank group in 100 μL/well, and the cell plate was incubated in a cell incubator at 37° C. and 5% of CO₂ for 48 hours. After the cells were incubated for 48 hours, the 96-well plate was centrifuged at 2000 rpm for 5 minutes. The cell supernatants were taken out, and the mouse cytokines IFN-γ and mouse IL-2 were assayed according to the instruction of Mouse IL-2 precoated ELISA kit or Mouse IFN-γ precoated ELISA kit. Absorbances at 450 nm (OD450) were detected by the light absorption mode of a microplate reader. The histogram of the levels of expression of IFN-γ and IL-2 by the splenocytes with the positive control PcAb (4.1D3) and the hTIGIT mouse-derived antibodies (m60H5 and m51C1) can be obtained by using the data processing software GraphPad Prism 5. The experimental results shown in FIGS. 7 and 8: the capabilities of the positive control PcAb (4.1D3 and the hTIGIT mouse-derived antibodies (m60H5 and m51C1) in inducing the T cells to secrete mIFN-γ and mIL-2 are different; all the mouse-derived antibodies can continuously induce the T cells to secrete mIFN-γ (FIG. 8), and in particular, m51C1 and m60H5 have very strong continuous induction activities; but the influence on the amount of secreted mIL-2 is not obvious (FIG. 7).

### Example 9: Secretion of Cytokines by Inducing T Cells of B-hTIGIT Transgenic Mice with hTIGIT Humanized Antibodies

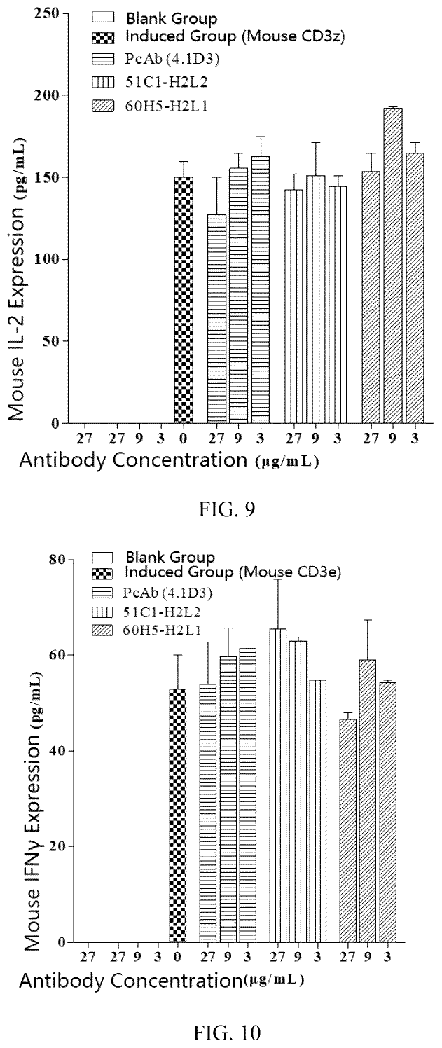

The treatment method in the present example was the same as that in example 8, except that the hTIGIT mouse-derived antibodies (m60H5 and m51C1) were replaced by the hTIGIT humanized antibodies (h60H5 and h51C1). The results shown in FIGS. 9 and 10: both the positive control PcAb (4.1D3) and the hTIGIT humanized antibodies (h60H5 and h51C1) can induce the T cells of the B-hTIGIT transgenic mice to secrete IFN-γ and IL-2, and the induction activities of the hTIGIT humanized antibodies (h60H5 and h51C1) are better than that of the positive control PcAb (4.1D3) at some concentrations.

### Example 10: Secretion of Cytokines by Inducing Jurkat Cells Activation with hTIGIT Mouse-Derived Antibodies

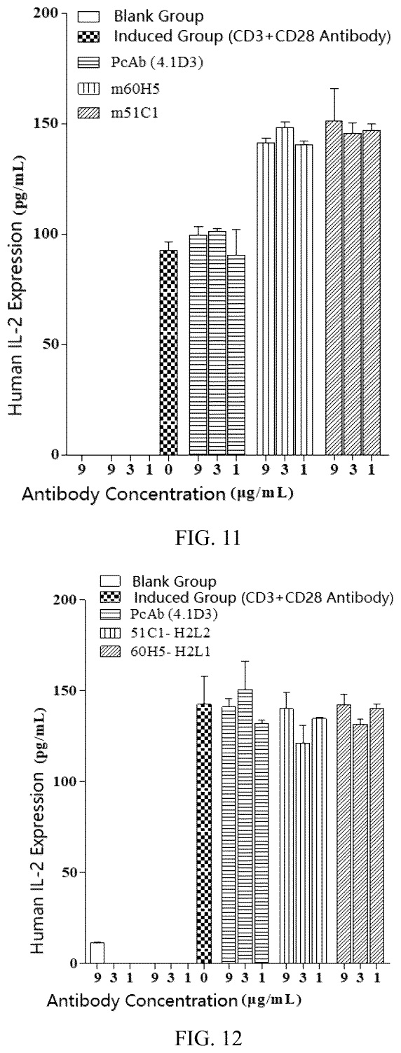

Jurkat cells were collected and counted. The cell density was adjusted to 0.8×10⁶ cells/mL, and the cells were plated into a 96-well plate in 0.04×10⁶ cells/well and 50 μL/well. The prepared corresponding antibodies (the hTIGIT mouse-derived antibodies (m60H5 and m51C1) and the positive control PcAb (4.1D3)) were added: the antibodies were diluted from an initial concentration of 13.5 μg/mL (i.e. 1.5 folds of the initial concentration), and three concentrations were obtained by gradient dilution in 1:3, i.e. 9 μg/mL, 3 μg/mL and 1 μg/mL. The diluents were 1640 complete medium (containing 10% of FBS) containing 0.375 μg/ml (1.5×) of CD3 protein and 0.1875 μg/ml (1.5×) of CD28 protein. The 1640 complete medium containing 0.375 μg/ml of CD3 protein and 0.1875 μg/ml of CD28 protein was added into the induced group, the 1640 complete medium was added into the blank group, and they were added into the cell wells in 100 μL/well and incubated in a cell incubator at 37° C. and 5% of CO₂ for 48 hours. After 48 hours, the 96-well plate was taken out and centrifuged at 2000 rpm for 5 minute, and the supernatants were taken out. The human cytokine IL-2 was assayed according to the instruction of Human IL-2 precoated ELISA kit. Absorbances at 450 nm (OD450) were detected by the light absorption mode of a microplate reader. The data processing software GraphPad Prism 5 was used to describe and analyze the levels of expression of human IL-2 by Jurkat cells stimulated by the positive control PcAb (4.1D3) and the hTIGIT mouse-derived antibodies in a histogram. The results shown in FIG. 11: both the hTIGIT mouse-derived antibodies (m60H5 and m51C1) and the positive control 4.1D3 can induce the activated Jurkat cells to continuously secrete human IL-2 cytokines.

### Example 11: Secretion of Cytokines by Inducing Jurkat Cells Activation with hTIGIT Humanized Antibodies

The treatment method in the present example was the same as that in example 10, except that the hTIGIT mouse-derived antibodies (m60H5 and m51C1) were replaced by the hTIGIT humanized antibodies (h60H5 and h51C1). The results shown in FIG. 12: both the hTIGIT humanized antibodies (h60H5 and h51C1) and the positive control 4.1D3 can induce the activated Jurkat cells to continuously secrete human IL-2 cytokines. Compared with the mouse-derived antibodies, the capability of each humanized antibody to induce the secretion of IL-2 is significantly enhanced, which may be associated with the human IgG1 Fc functional domain.

### Example 12: Secretion of Cytokines by Inducing Human PBMC Cells with hTIGIT Mouse-Derived Antibodies

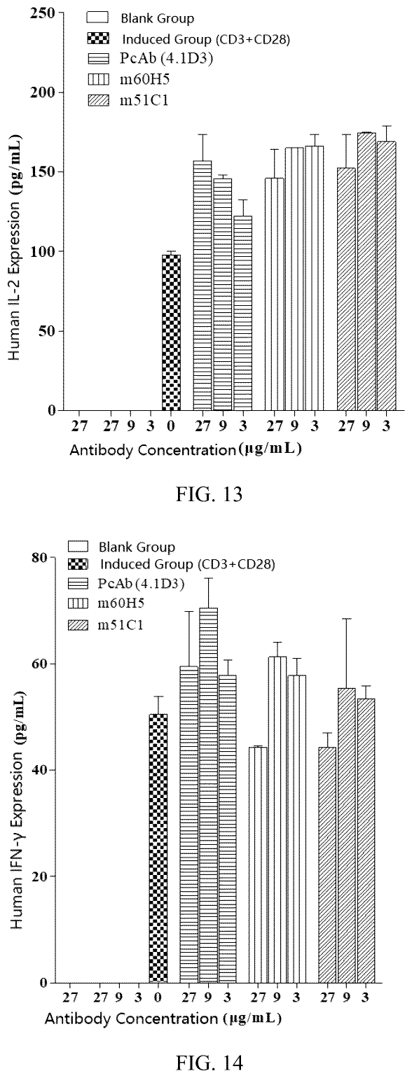

Purified PBMC cells were counted. The cell density was adjusted to 2×10⁶ cells/mL, and the cells were plated into a 96-well plate in 0.1×10⁶ cells/well and 50 μL/well. The prepared corresponding antibodies (the hTIGIT mouse-derived antibodies or the positive control PcAb (4.1D3)) were added: the antibodies were diluted from an initial concentration of 40.5 μg/mL (1.5×), and three concentrations were obtained by gradient dilution in 1:3. The diluents were DMEM complete medium containing 0.75 μg/mL (1.5×) of CD3 protein and 0.375 μg/mL (1.5×) of CD28 protein. Only the complete DMEM medium containing 0.75 μg/mL of CD3 protein and 0.375 μg/mL of CD28 protein was added into the induced group, the complete DMEM medium was added into the blank group in 100 μL/well. The cells were incubated in a cell incubator at 37° C. and 5% of CO₂ for 24 hours. After 24 hours, the cell culture plate was centrifuged at 2000 rpm for 5 minutes. The cell supernatants were taken out, and human IFN-γ and human IL-2 were assayed according to the instruction of Human IL-2 precoated ELISA kit or Human IFN-γ precoated ELISA kit. Absorbances at 450 nm (OD450) were detected by the light absorption mode of a microplate reader. The data processing software GraphPad Prism 5 was used to describe and analyze the levels of expression of human IFN-γ and human IL-2 induced by the positive control PcAb (4.1D3) and the hTIGIT mouse-derived antibodies in a histogram. The capabilities of the hTIGIT mouse-derived antibodies and the positive control 4.1D3 to induce the human PBMC cells co-stimulatorily activated by CD3 and CD28 to continuously secrete human IFNγ and IL-2 were assayed. The results shown in FIGS. 13 and 14: both the hTIGIT mouse-derived antibodies and the positive control 4.1D3 can induce the continuous secretion of IL-2 (FIG. 13), but the induction of the level of secretion of human IFN-γ is not significant (FIG. 14).

### Example 13: Secretion of Cytokines by Inducing Human PBMC Cells with hTIGIT Humanized Antibodies

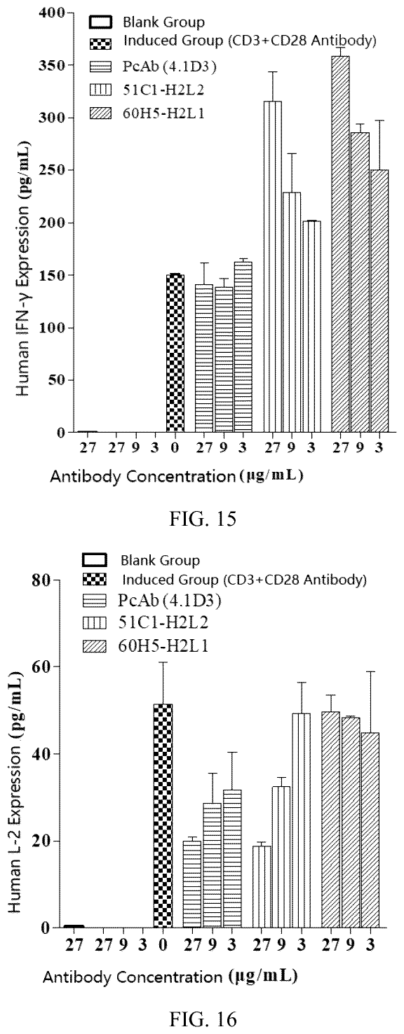

The treatment method in the present example was the same as that in example 12, except that the hTIGIT mouse-derived antibodies (m60H5 and m51C1) were replaced by the hTIGIT humanized antibodies (h60H5 and h51C1). The results shown in FIGS. 15 and 16: the hTIGIT humanized antibodies (h60H5 and h51C1) can induce the continuous secretion of IFN-γ (FIG. 15), showing the continuous capability to induce the continuous secretion of human IL-2.

### Example 14: Analysis of Competition Between hTIGIT Mouse-Derived Antibodies and 4.1D3 for hTIGIT Epitopes

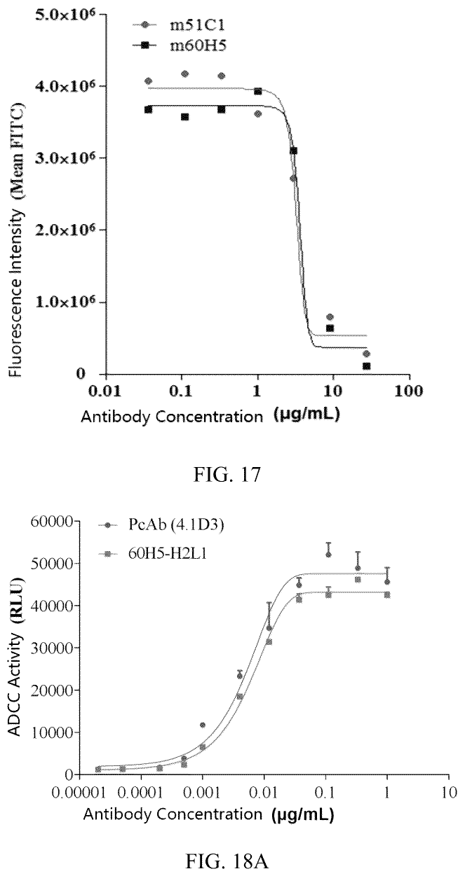

Cells were collected and counted. The 293F-hTIGIT-short #1B1 cell line was digested, terminated, centrifuged, resuspended and counted, and the cell density was adjusted to 4×10⁶ cells/mL, in 50 μL/tube, a total of 20×10⁴ cells were added into 1.5 mL EP tubes. The prepared hTIGIT mouse-derived antibodies (m60H5 and m51C1) were added. Both the hTIGIT mouse-derived antibodies (m60H5 and m51C1) were diluted to an initial concentration of 108 μg/mL. Seven concentrations were obtained by gradient dilution in 1:3, and added into the corresponding tubes in 50 μL/tube. The blank group, the PcAb (4.1D3) group and the hTIGIT mouse-derived antibody group were added with 50 μL of PBS respectively, gentle blowing and mixing were performed, and incubation was performed on ice for 15 minutes first. After 15 minutes, the prepared PcAb (4.1D3) antibody (6 μg/ml) was added into the hTIGIT mouse-derived antibody group and the PcAb (4.1D3) group in 100 μL/tube, 100 μL of PBS was added into the blank group, gentle blowing and mixing were performed, and incubation was then performed on ice for 1 hour. After incubation, 500 μL of PBS was added for centrifugal washing once, centrifugation was performed at 1000 rpm for 5 minutes, and the supernatants were discarded. A fluorescent secondary antibody was added (an Alexa® 488 fluorescently labeled anti-human IgG antibody was diluted with PBS (1:500) in 200 μL/tube, gentle blowing and mixing were performed, and incubation was performed on ice in the dark for 30 minutes). After 30 minutes of incubation, they were loaded for assay. The samples were loaded for assay by using a flow cytometer. The hTIGIT epitope binding of the hTIGIT mouse-derived antibodies and PcAb (4.1D3) was analyzed, so that the competitive binding conditions of m60H5 and m51C1 were obtained respectively, and the IC50 values of both were calculated. The competitive binding activities of the hTIGIT mouse antibody and the 4.1D3 antibody (human IgG1 subtype) with hTIGIT epitopes were analyzed, so that it was determined that the screened hTIGIT mouse-derived antibodies (m60H5 (IC50=5.02 μg/mL) and m51C1 (IC50=4.08 μg/mL)) competed with the positive control PcAb (4.1D3) for binding with the same epitopes. The results shown in FIG. 17: it can be seen from the flow binding peak graph and the fluorescence intensity-concentration dose relationship that the screened hTIGIT mouse-derived antibodies (m60H5 and m51C1) and the 4.1D3 antibody compete for the same or very close epitopes of hTIGIT.

### Example 15: ADCC Effects of hTIGIT Humanized Antibodies

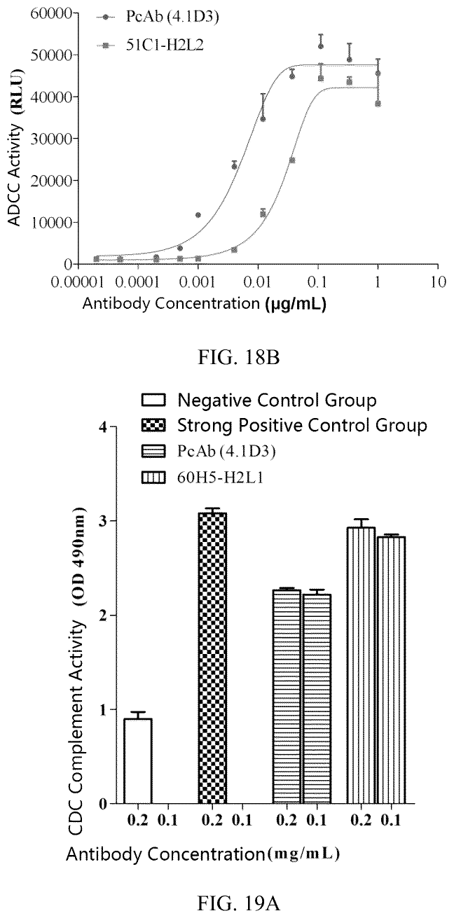

293F-TIGTI-short #1B1 cells and Jurkat-NFAT-Luc2-CD16a-V158 cells (purchased from Kyinno Bio, #KC-1507) were prepared. The cells were counted, and the cell densities were adjusted to 0.4×10⁶ cells/mL. The two cell lines were uniformly mixed according to an equal volume of 1:1, and the mixed cells were plated into a 96-well white plate (opaque) in 50 μL/well, i.e. 0.02×10⁶ cells/well for each of the two cell lines. The prepared corresponding antibodies (the hTIGIT humanized antibodies (h60H5 and h51C1) or the positive control PcAb (4.1D3)) were added: the antibodies were diluted from an initial concentration of 2 μg/mL (2×). Eleven concentrations were obtained by gradient dilution in 1:3, the diluents were 1640 complete medium (containing 10% of FBS). The 1640 complete medium was added into the blank group according to 50 μL/well, and the cells were incubated in a cell incubation at 37° C. and 5% of CO₂ for 6 hours. After 6 hours of co-incubation, the 96-well white plate was taken out and equilibrated at room temperature for 15 minutes. Luciferase substrate reagent was then added in 50 μL/well, and the reaction was conducted at room temperature in the dark for 5 minutes. Chemiluminescence signals were detected by the luminescent mode of a microplate reader. Four-parameter curves of fluorescence signals of the positive control PcAb (4.1D3) and the hTIGIT humanized antibodies can be obtained by using data processing software, so that the EC50 values of antigen-antibody dependent cell killing effects can be obtained. A chemiluminiscent reporter gene method was used to simulate the antibody-dependent cell killing effect of T cells, NK cells or the like. The ADCC effect was embodied by constructing an exogenously expressed FcγRIII (CD16) receptor and a downstream NFAT transcription factor thereof in Jurkat cells and a binding domain of the transcription factor and a luciferase reporter gene capable of being transcribed. When the constructed Jurkat-NFAT-Luc2-CD16a cells were co-incubated with 293F-TIGIT-short expressing hTIGIT, the antibodies could activate the signal transduction pathway of the Jurkat-NFAT-Luc2-CD16a cells under the mediation of the antibodies. The experimental results shown in FIG. 18A-B: both the hTIGIT humanized antibodies and the positive control PcAb (4.1D3) exhibit significant ADCC effects, and the non-specific antibody group does not exhibit ADCC effect; in addition, the ADCC effect of h60H5 is better than that of h51C1; and the ADCC effect of h60H5 is equivalent to that of the positive control 4.1D3.

### Example 16: CDC Effects of hTIGIT Humanized Antibodies

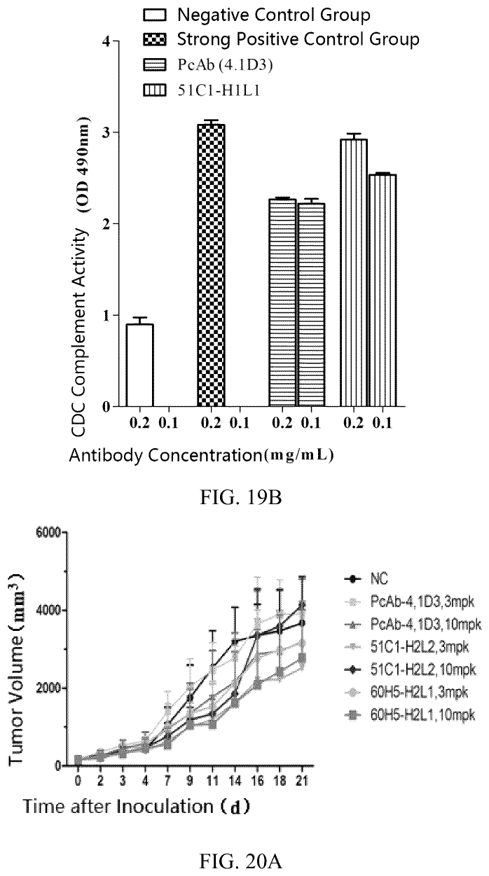

293F-hTIGIT-short #1B1 cells were collected and counted, the cell density was adjusted to 0.5×10⁶ cells/mL, and the cells were plated into a 96-well plate in 40 μL/well, that is, the cell number was 0.02×10⁶ cells/well. The prepared corresponding antibodies (the hTIGIT mouse-derived antibodies (h60H5 and h51C1) and the positive control PcAb (4.1D3)) were added: the antibodies were diluted from an initial concentration of 400 μg/mL (2×), two concentrations were obtained by gradient dilution in 1:2, and the diluents were Freestyle 293 complete medium (containing 5% of FBS). The negative control group and the strong positive control group were Freestyle 293 complete medium (containing 5% of FBS) (the strong positive control group was added with a cell lysis reagent prepared by Cytotoxicity LDH Assay Kit as a means of cell death). The antibody group, the negative control group and the strong positive control group were all added in 50 μL/well and have 3 replicate wells. The cells were then incubated in a cell incubator at 37° C. and 5% of CO₂ for 0.5 hour. After 0.5 hour, fresh human serum was added in 10 μL/well, and the cells were then incubated in a cell incubator at 37° C. and 5% of CO₂ for 3 hours. After 0.5 hours of incubation, the strong positive control group was added with Lysis Buffer in Cytotoxicity LDH Assay Kit in 10 μL/well, and the cells were incubated in the cell incubator at 37° C. and 5% of CO₂ for 0.5 hour. After 0.5 hour, the prepared Working Solution in Cytotoxicity LDH Assay Kit was added in 100 μL/well, and reaction was conducted at room temperature in the dark for 30 minutes. Stop Solution in Cytotoxicity LDH Assay Kit was added in 50 μL/well, and absorbances (OD490) at 490 nm were immediately detected with the light absorption mode of a microplate reader. The data processing software GraphPad Prism 5 was used to analyze LDH (serum lactate dehydrogenase) levels mediated by the negative control group, the strong positive control group, the positive control group and the hTIGIT humanized antibodies in a histogram. When carrying out the complement-dependent antibody killing effect (CDC), fresh human anticoagulant plasma was needed in order to ensure the complement activity in the plasma. The experimental results shown in FIG. 19A-B: that the hTIGIT humanized antibodies (h60H5 and h51C1) and the positive control PcAb have CDC killing effect on the cells expressing hTIGIT, and the effect of the hTIGIT humanized antibodies (h60H5 and h51C1) is better than that of the positive control PcAb.

### Example 17: Experiment of Efficacy of hTIGIT Humanized Antibodies on Colon Cancer Cell Line CT26 Tumor-Bearing Mice

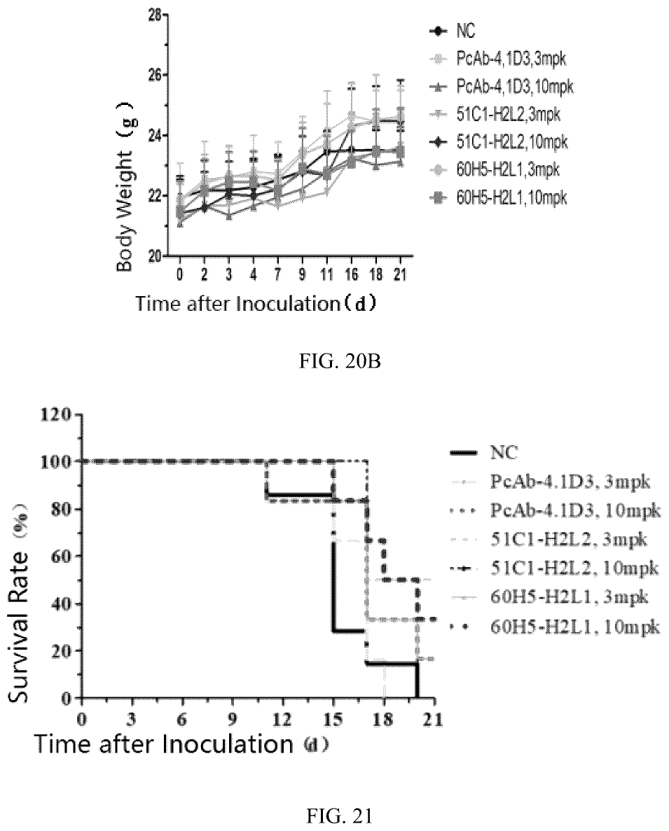

The efficacies of the antibodies were studied by using adult Balb/c-huTIGIT (B-huTIGIT) transgenic female mice (purchased from GemPharmatech, Nanjing, China) aged 8 to 12 weeks. After being purchased, the transgenic mice were observed and reared in an SPF animal house. The transgenic mice were adaptively fed for one week at a constant temperature of (23±2)° C. and a humidity of 40% to 60% RH in rearing rooms with 12 hours of artificial light illumination and 12 hours of dark, and ate Co60 radiation feed and tap water by themselves. Balb/c mouse-derived colon cancer cells CT26. WT which grew well were digested and collected, and the density of single cell suspension was adjusted to 1×10⁷ cells/mL. Each mouse was injected with 1×10⁶ cells/100 μL/mouse by subcutaneous injection (s.c.) at the armpit. After the tumor cells were inoculated, the mice continued to be reared, and the body weights and tumor volumes of the mice were continuously monitored. When the tumor volume reached 80 to 120 mm³, the mice became a successful tumor-bearing mouse model. The mice that had successfully borne tumors were randomly divided into groups. According to the experimental requirements, a negative control group, a positive control group and a to-be-tested drug experiment group were set respectively. The mice in each group were weighed, the drug group was set as a high-dose group and a low-dose group respectively, and intraperitoneal injection was then performed for administration. The changes of the body weights and tumor volumes of the mice were continuously monitored during administration until the end of the experiment. General condition, mental state, activities, bleeding and ulceration at the inoculation site and other conditions were closely observed, and the abnormal conditions of the mice were photographed and recorded. When the tumor volume of the mice reached 3000 mm³ or the mice were very thin and weak and their life state was poor, it was regarded as the experimental endpoint of the mice, and the mice needed to be killed by cervical dislocation. When the experimental endpoint was reached, the mice were killed by cervical dislocation. The tumors were then resected, photographed and weighed, and efficacy (tumor size)-drug concentration curves, mouse body weight change curves and survival curves were drawn. After the colon cancer cell CT26 tumor-bearing transgenic mice B-huTIGIT were randomly divided into groups, a negative control group, a positive antibody (PcAb (4.1D3)) control group and a humanized antibody group were set. 10 mg/kg and 3 mg/kg were set for each antibody group. The tumor-bearing mice were treated by intraperitoneal administration. The results shown in FIG. 20A-B: the hTIGIT humanized antibodies (h60H5 and h51C1) and the positive control PcAb (4.1D3) can effectively decrease the tumor growth rate (FIG. 20A), and there was no significant difference in the changes of the body weights of the mice in each group during the experiment (FIG. 20B). The experimental mice that reached the experimental endpoint were killed by cervical dislocation, and the survival curves of the mice were drawn by Prism 5.0 software. The results shown in FIG. 21: it can be seen from the treatment-survival curves that both the high- and low-hTIGIT humanized antibody h51C1 dose groups and the high-h60H5 dose group can effectively increase the survival rate of the mice.

### Example 18: Tumor Re-Challenge Experiment on Tumor-Bearing Cured Mice

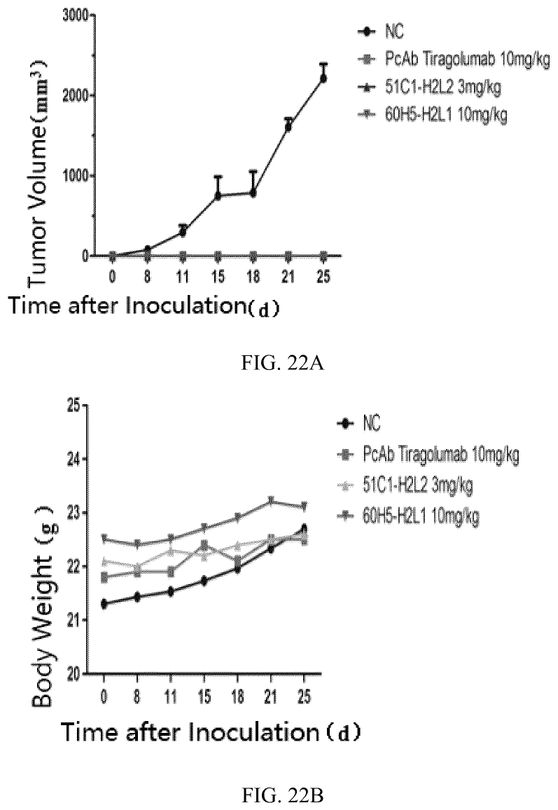

During the experiment on the efficacy of the hTIGIT humanized antibodies on the tumor-bearing transgenic mice in example 17, it was discovered that the three groups of mice treated by the positive control PcAb (4.1D3) (10 mg/kg), the TIGIT humanized antibody h51C1 (3 mg/kg) and the TIGIT humanized antibody h60H5 (10 mg/kg) all showed the phenomenon of tumor regression or even tumor disappearance. Considering that the TIGIT antibodies have the function of inducing the body to produce immune memory, in order to verify this guess, it was designed to re-inoculate colon cancer cell line CT26.WT to the mice with tumor regression, and the method of re-inoculating the CT26 cells was the same as that described in example 17. At the same time, a blank control group was set, and axillary subcutaneous injection was performed in 1×10⁶ cells/100 μL/mouse (at the other side not incubated with tumor cells), and treatment was then performed according to the dosage and frequency. The growth of tumors in the mice and the change of the body weights of the mice were observed and recorded. The experimental results shown in FIG. 22A-B: from day 8 to day 25 after the injection of CT 26. WT, the tumors of the blank control group continued to grow until they exceeded 2000 mm³; and the tumors of the antibody therapy group stopped growing (FIG. 22A), and there was no significant difference in the body weights of the mice in each group (FIG. 22B).

### Example 19: Experiment of Efficacy of hTIGIT Humanized Antibodies on Breast Cancer Cell Line 4T1 Tumor-Bearing Mice

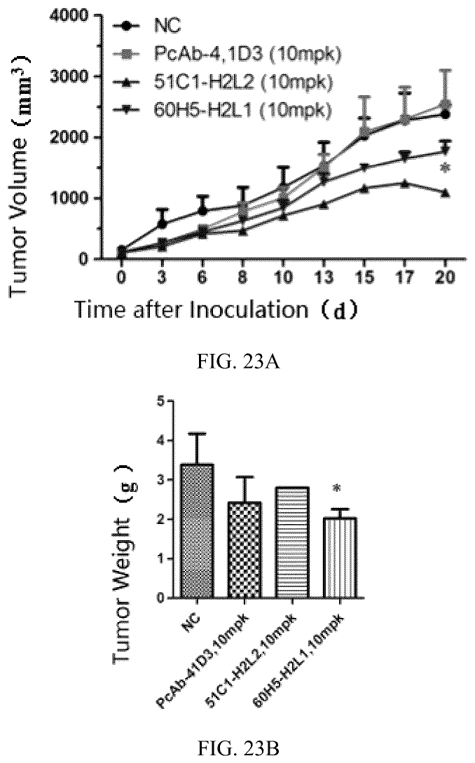

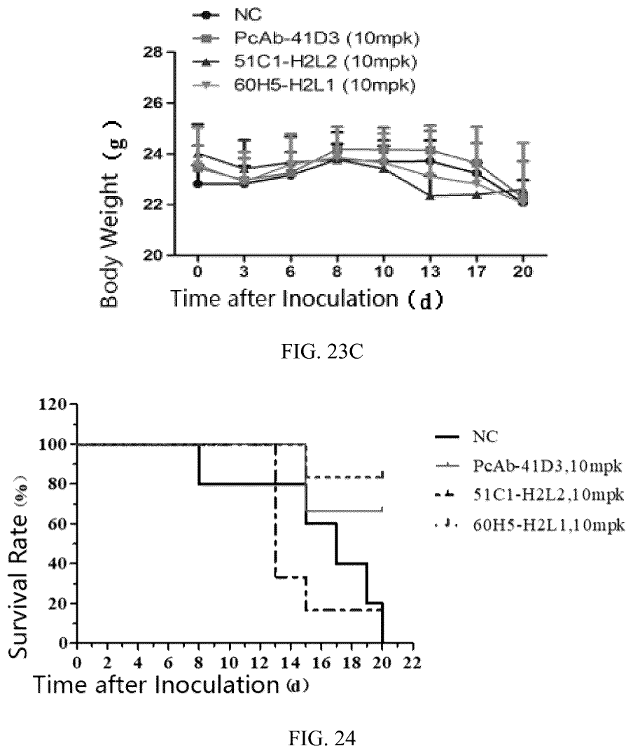

The method of bearing breast cancer cell 4T1 tumors in the B-huTIGIT transgenic mice was the same as that in example 17. After the tumor-bearing transgenic mice B-huTIGIT were randomly divided into groups, a negative control group, a positive antibody control group and a humanized antibody group were set. 10 mg/kg and 3 mg/kg were set for each antibody group. After the tumor-bearing mice were treated by intraperitoneal administration, the results shown in FIG. 23A-C: the tumor volume of the positive control group PcAb (4.1D3) continued to increase, and there was no difference between the positive control group and the blank control group; the tumor volume of the humanized antibody h51C1 group began to decrease from day 13; and the tumor volume (size) of the h60H5 group was significantly reduced on day 20. The antibody therapy experiment reached the experimental endpoint on day 21, and the tumor tissues were separated out after the mice were killed. The tumor volume at the endpoint showed that the antibody therapy group h60H5 had the best effect in inhibiting the proliferation of the 4T1 tumor (FIG. 23B, P<0.05). According to the above analysis, the TIGIT humanized antibodies (h51C1 and h60H5) can decrease the tumor growth rate. There was no significant difference in the weights of the mice in each group during the experiment (FIG. 23C). Each hTIGIT antibody can significantly prolong the survival time of the 4T1 tumor-bearing mice (FIG. 24).

### Example 20: Pharmacokinetic Assay of hTIGIT Humanized Antibodies

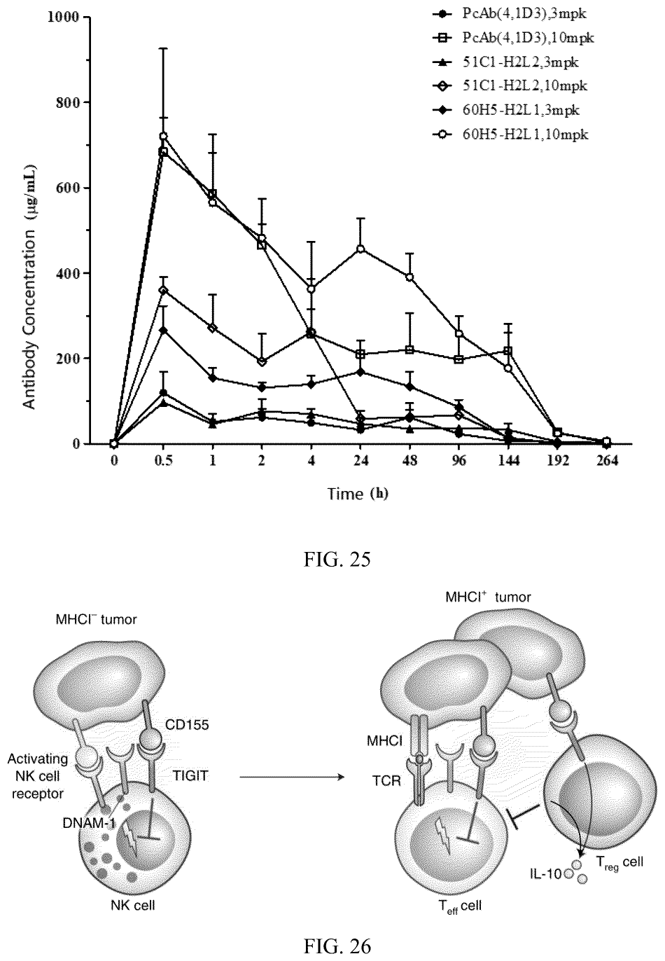

Adult Balb/c mice (regardless of gender) aged 8 to 12 weeks were used to study the in-vivo metabolism of the antibodies. After being purchased, the mice were observed and reared in SPF animal house. The transgenic mice were adaptively fed for one week at a constant temperature of (23±2)° C. and a humidity of 40% to 60% RH in rearing rooms with 12 hours of artificial light illumination and 12 hours of dark, and ate Co60 radiation feed and tap water by themselves. Before administration, the mice were weighed, blood was collected (DO), and the mice were divided into groups and dose volumes were calculated according to the body weights of the mice. According to the experimental requirements, a positive control group (PcAb (4.1D3)) and a to-be-tested drug experiment group (TIGIT humanized antibodies (h51C1 and h60H5)) were set. Intravenous administration was performed according to the relationship between the body weights of the mice and dosages (10 mg/kg (mpk) and 3 mg/kg (mpk) were set for each antibody group). 30 minutes, 1 hours, 2 hours, 4 hours, 24 hours, 48 hours, 4 days, 8 days and 11 days after administration, blood was collected in the form of retro-orbital venous plexus in 100 μL/mouse each time, and the blood was allowed to stand at room temperature for 30 minutes, centrifugation was performed at 4000 rpm for 10 minutes, and the serum was taken out. When the plasma concentration at the last blood collection point was 1/20 of the highest plasma concentration, blood collection was stopped. The plasma concentration was assayed by indirect ELISA. The metabolism of the positive control group and the humanized antibodies in the mice was assayed. That is, 0.5 μg/mL of TIGIT-his antigen was coated, and the to-be-tested serum diluent was added. After the absorbances were finally detected chromogenically, four-parameter curves of the positive control (PcAb (4.1D3)) and the candidate humanized antibodies (h51C1 and h60H5) were obtained by data processing software. EC50 values were obtained, and the concentration (Cmax), half-life (T½) and area under the curve AUC of each group at each time point were calculated. It can be seen from the metabolic curves of the antibody drugs that each hTIGIT antibody had relatively good pharmacokinetic activity in the mice, and there was no sudden disappearance of antibody concentration, and the concentration in the blood showed a gentle downtrend (FIG. 25). The data processing software PK Solution 2.0 was used to calculate the main pharmacokinetic parameters of the positive control 4.1D3 group, the humanized antibody h51C1 group and the humanized antibody h60H5 group (e.g. area under the plasma concentration-time curve AUC (0-t), area under the plasma concentration-time curve AUC (0-0), peak concentration (Cmax), peak time (Tmax), elimination half-life (T½), apparent volume of distribution (Vd) and clearance rate (CL). The experimental study results are shown in Table 2. The drug concentrations of h51C1 and h60H5 showed initial concentration-dependent plasma concentration changes, and the blood antibody concentrations decreased to 1/20 or less of the highest value after day 11.

TABLE 2
Mean (Mean ± SD) of Main Pharmacokinetic Parameters of Each TIGIT Humanized Antibody Dosage Groups

| | AUC(0-t) | AUC(0-∞) | Cₘₐₓ | Tₘₐₓ | T_{1/2} | Vd | CL |
|---|---|---|---|---|---|---|---|
| Group | (μg/L*h) | (μg/L*h) | (mg/L) | (h) | (h) | (mL/kg) | (L/h/kg) |
| PcAb(4.1D3), 3 mpk | 4364.5 ± 2207.9 | 4808.2 ± 2747.6 | 123.7 ± 105.4 | 1.1 ± 0.8 | 56.4 ± 13.3 | 71.8 ± 32.3 | 0.9 ± 0.6 |
| PcAb(4.1D3), 10 mpk | 41020.6 ± 12017.1 | 37746.1 ± 14458.1 | 3800.3 ± 4408.4 | 0.4 ± 0.4 | 45 ± 21.5 | 14.7 ± 4.4 | 0.3 ± 0.1 |
| 51C1-H2L2, 3 mpk | 6934.6 ± 2146.5 | 7121.2 ± 2402.2 | 121.8 ± 30.7 | 1.5 ± 1.5 | 59.5 ± 17.6 | 34.4 ± 8.0 | 0.5 ± 0.2 |
| 51C1-H2L2, 10 mpk | 10364.4 ± 4203.1 | 10364.4 ± 4203.1 | 458.2 ± 174.2 | 1.2 ± 1.6 | 29.7 ± 11.8 | 44.2 ± 10.2 | 1.1 ± 0.5 |
| 60H5-H2L1, 3 mpk | 15492.6 ± 5473.1 | 15492.6 ± 5473.1 | 1035.2 ± 1175.5 | 0.1 ± 0.2 | 27.8 ± 8.3 | 10.1 ± 7.1 | 0.2 ± 0.1 |
| 60H5-H2L1, 10 mpk | 65209.4 ± 23912.4 | 65792.2 ± 24170.6 | 992.34 ± 477.9 | 1.6 ± 1.5 | 55.7 ± 15.9 | 13.8 ± 5.8 | 0.2 ± 0.1 |

The above-mentioned examples are preferred embodiments of the present disclosure, but the embodiments of the present disclosure are not limited by the above-mentioned examples, and any other alterations, modifications, replacements, combinations and simplifications which are made without departing from the spirit and principle of the present disclosure should all be equivalent replacement patterns, and should all be included in the scope of protection of the present disclosure.

1. A human TIGIT antibody, 60H5 or 51C1, or an antigen-binding fragment thereof,
the 60H5 comprises a 60H5 heavy chain variable domain and a 60H5 light chain variable domain;
the 60H5 heavy chain variable domain comprises CDR1, CDR2, and CDR3;
the amino acid sequence of CDR1 of the 60H5 heavy chain variable domain comprises:
a) GYTFTEYT (SEQ ID NO.: 2); or
b) a sequence having 95%, 96%, 97%, 98% or 99% sequence identity with the amino acid sequence shown as SEQ ID NO.: 2;
the amino acid sequence of CDR2 of the 60H5 heavy chain variable domain comprises:
a) INPNNGGT (SEQ ID NO.: 3); or
b) a sequence having 95%, 96%, 97%, 98% or 99% sequence identity with the amino acid sequence shown as SEQ ID NO.: 3;
the amino acid sequence of CDR3 of the 60H5 heavy chain variable domain comprises:
a) ARSGNWDYAMDY (SEQ ID NO.: 4); or
b) a sequence having 95%, 96%, 97%, 98% or 99% sequence identity with the amino acid sequence shown as SEQ ID NO.: 4;
the 60H5 light chain variable domain comprises CDR1, CDR2, and CDR3;
the amino acid sequence of CDR1 of the 60H5 light chain variable domain comprises:
a) QHVSTA (SEQ ID NO.: 7); or
b) a sequence having 95%, 96%, 97%, 98% or 99% sequence identity with the amino acid sequence shown as SEQ ID NO.: 7;
the amino acid sequence of CDR2 of the 60H5 light chain variable domain comprises:
a) SAS (SEQ ID NO.: 8); or
b) a sequence having 95%, 96%, 97%, 98% or 99% sequence identity with the amino acid sequence shown as SEQ ID NO.: 8;
the amino acid sequence of CDR3 of the 60H5 light chain variable domain comprises:
a) QQHYITPWT (SEQ ID NO.: 9); or
(b) a sequence having 95%, 96%, 97%, 98% or 99% sequence identity with the amino acid sequence shown as SEQ ID NO.: 9;
51C1 comprises a 51C1 heavy chain variable domain and a 51C1 light chain variable domain;
the 51C1 heavy chain variable domain comprises CDR1, CDR2, and CDR3;
the amino acid sequence of CDR1 of the 51C1 heavy chain variable domain comprises:
a) GYTFTEYF (SEQ ID NO.: 12); or
b) a sequence having 95%, 96%, 97%, 98% or 99% sequence identity with the amino acid sequence shown as SEQ ID NO.: 12;
the amino acid sequence of CDR2 of the 51C1 heavy chain variable domain comprises:
a) FYPGSGSI (SEQ ID NO.: 13); or
b) a sequence having 95%, 96%, 97%, 98% or 99% sequence identity with the amino acid sequence shown as SEQ ID NO.: 13;
the amino acid sequence of CDR3 of the 51C1 heavy chain variable domain comprises:
a) ARHEMRYGNYVLDY (SEQ ID NO.: 14); or
b) a sequence having 95%, 96%, 97%, 98% or 99% sequence identity with the amino acid sequence shown as SEQ ID NO.: 14;
the 51C1 light chain variable domain comprises CDR1, CDR2, and CDR3;
the amino acid sequence of CDR1 of the 51C1 light chain variable domain comprises:
a) TGAVTTRNY (SEQ ID NO.: 17); or
b) a sequence having 95%, 96%, 97%, 98% or 99% sequence identity with the amino acid sequence shown as SEQ ID NO.: 17;
the amino acid sequence of CDR2 of the 51C1 light chain variable domain comprises:
a) GTN (SEQ ID NO.: 18); or
b) a sequence having 95%, 96%, 97%, 98% or 99% sequence identity with the amino acid sequence shown as SEQ ID NO.: 18;
the amino acid sequence of CDR3 of the 51C1 light chain variable domain comprises:
a) GLWYSNHLV (SEQ ID NO.: 19); or
b) a sequence having 95%, 96%, 97%, 98% or 99% sequence identity with the amino acid sequence shown as SEQ ID NO.: 19.

2. The human TIGIT antibody or the antigen-binding fragment thereof according to claim 1, wherein:
the amino acid sequence of the 60H5 heavy chain variable domain comprises:
a) EVQLQQSGPELVKPGASLKISCKTSGYTFTEYTMHWVKQSHGKSLEWIGGINPN NGGTKFKGKATLTVDKSSSTAYMELRSLTSEDSAVYYCARSGNWDYAMDYWGQGTSVT VSS (SEQ ID NO.: 1); or
b) a sequence having 95%, 96%, 97%, 98% or 99% sequence identity with the amino acid sequence shown as SEQ ID NO.: 1; or
c) QVQLVQSGAEVKKPGASVKVSCKTSGYTFTEYTMHWVRQAPGQRLEWIGGINP NNGGTSYNQKFQGRVTITVDTSASTAYMELSSLRSEDTAVYYCARSGNWDYAMDYWG QGTTVTVSS (SEQ ID NO.: 21); or
d) a sequence having 95%, 96%, 97%, 98% or 99% sequence identity with the amino acid sequence shown as SEQ ID NO.: 21;
the amino acid sequence of the 60H5 light chain variable domain comprises:
a) DIVMTQSHKFMSTSVGDRVSITCKASQHVSTAVVWYQQKPGQSPKLLIYSASYRY TGVDRFTGSGSGTDFTFTISSVQAEDLAVYYCQQHYITPWTFGGGTKLEIKRADA (SEQ ID NO.: 6); or
b) a sequence having 95%, 96%, 97%, 98% or 99% sequence identity with the amino acid sequence shown as SEQ ID NO.: 6; or
c) DIQMTQSPSSMSASVGDRVTITCKASQHVSTAVVWYQQKPGKAPKLLIYSASYRY TGVPDRFSGSGSGTDFTFTISSLQPEDIATYYCQQHYITPWTFGGGTKLEIKRTVA (SEQ ID NO.: 22); or
d) a sequence having 95%, 96%, 97%, 98% or 99% sequence identity with the amino acid sequence shown as SEQ ID NO.: 22;
the amino acid sequence of the 51C1 heavy chain variable domain comprises:
a) QVQLQQSGAELVKPGASVKLSCKASGYTFTEYFIHWIKQKSGQGLEWIGWFYPG SGSIKYNERFKDKATLTADKSSSTVYMELSRLTSEDSAVYFCARHEMRYGNYVLDYWGQ GTTLTVSS (SEQ ID NO.: 11); or
b) a sequence having 95%, 96%, 97%, 98% or 99% sequence identity with the amino acid sequence shown as SEQ ID NO.: 11; or
c) QVQLVQSGAEVKKPGASVKVSCKASGYTFTEYFIHWVRQAPGQGLEWIGWFYP GSGSIKYNERFKDRVTLTADTSISTAYMELSRLRSDDTAVYYCARHEMRYGNYVLDYWG QGTTVTVSS (SEQ ID NO.: 23); or
d) a sequence having 95%, 96%, 97%, 98% or 99% sequence identity with the amino acid sequence shown as SEQ ID NO.: 23;
the amino acid sequence of the 51C1 light chain variable domain comprises:
a) QAVVTQESALTTSPGETVTLTCRSSTGAVTTRNYANWVQEKPDHLFTGLIGGTNN RVPGVPARFSGSLIGDKAALTITGAQTEDEAIYFCGLWYSNHLVFGGGTKLTVLGQPK (SEQ ID NO.: 16); or
b) a sequence having 95%, 96%, 97%, 98% or 99% sequence identity with the amino acid sequence shown as SEQ ID NO.: 16; or
c) QAVVTQEPSLTVSPGGTVTLTCRSSTGAVTTRNYANWVQQKPGQAPRGLIGGTN NRVPGVPARFSGSLLGGKAALTLSGAQPEDEAEYYCGLWYSNHLVFGGGTKLTVLGQPK A (SEQ ID NO.: 24); or
d) a sequence having 95%, 96%, 97%, 98% or 99% sequence identity with the amino acid sequence shown as SEQ ID NO.: 24.

3. A nucleic acid molecule encoding the human TIGIT antibody or the antigen-binding fragment thereof according to claim 1.

4. An expression cassette, a recombinant vector or a transgenic cell line containing the nucleic acid molecule according to claim 3.

5. An immunoconjugate, comprising the human TIGIT antibody or the antigen-binding fragment thereof according to claim 1 and a conjugated portion,
the conjugated portion is at least one of a detectable marker, a drug, a toxin, a cytokine, an antibody, an antibody Fc fragment, an antibody scFv fragment, a radionuclide, an enzyme, a gold nanoparticle/nanorod, a magnetic nanoparticle, and a virus coat protein.

6. (canceled)

7. (canceled)

8. A product, comprising at least one of the human TIGIT antibody or the antigen-binding fragment thereof according to claim 1 and an immunoconjugate according to claim 5;
wherein the immunoconjugate comprises the human TIGIT antibody or the antigen-binding fragment thereof according to claim 1 and a conjugated portion,
the conjugated portion is at least one of a detectable marker, a drug, a toxin, a cytokine, an antibody, an antibody Fc fragment, an antibody scFv fragment, a radionuclide, an enzyme, a gold nanoparticle/nanorod, a magnetic nanoparticle, and a virus coat protein,
wherein the product is at least one of a reagent, an assay plate, and a kit.

9. A pharmaceutical composition, comprising at least one of (1) to (4);
(1) the human TIGIT antibody or the antigen-binding fragment thereof;
(2) a nucleic acid molecule encoding the human TIGIT antibody or the antigen-binding fragment thereof;
(3) an expression cassette, recombinant vector or transgenic cell line containing an nucleic acid molecule, wherein the nucleic acid molecule encoding the human TIGIT antibody or the antigen-binding fragment thereof, and
(4) an immunoconjugate comprising the human TIGIT antibody or the antigen-binding fragment thereof and a conjugated portion, wherein the conjugated portion is at least one of a detectable marker, a drug, a toxin, a cytokine, an antibody, an antibody Fc fragment, an antibody scFv fragment, a radionuclide, an enzyme, a gold nanoparticle/nanorod, a magnetic nanoparticle, and a virus coat protein,
wherein the human TIGIT antibody or the antigen-binding fragment thereof comprises the human TIGIT antibody or the antigen-binding fragment thereof according to claim 1.

10. The pharmaceutical composition according to claim 9, wherein
the conjugated portion of the immunoconjugate is a drug, a toxin, and/or a therapeutic isotope.

11. The pharmaceutical composition according to claim 10, wherein the pharmaceutical composition further comprises a pharmaceutically acceptable carrier.

12. A method for treating a tumor, comprises: administering a therapeutic amount of the human TIGIT antibody or the antigen-binding fragment thereof according to claim 1 to a subject in need.

13. A method for treating a tumor, comprises: administering a therapeutic amount of the expression cassette, recombinant vector or transgenic cell line according to claim 4 to a subject in need.

14. A method for treating a tumor, comprises: administering a therapeutic amount of the immunoconjugate according to claim 5 to a subject in need.

15. The method for treating a tumor according to claim 12, wherein the tumor comprises hematological neoplasms, solid tumors, non-small cell lung cancer, small cell lung cancer, colorectal cancer, melanoma, breast cancer, esophageal cancer, gastric tumors, bladder cancer, endometrial cancer, head and neck cancer, and renal cancer.

16. The method for treating a tumor according to claim 13, wherein the tumor comprises hematological neoplasms, solid tumors, non-small cell lung cancer, small cell lung cancer, colorectal cancer, melanoma, breast cancer, esophageal cancer, gastric tumors, bladder cancer, endometrial cancer, head and neck cancer, and renal cancer.

17. The method for treating a tumor according to claim 14, wherein the tumor comprises hematological neoplasms, solid tumors, non-small cell lung cancer, small cell lung cancer, colorectal cancer, melanoma, breast cancer, esophageal cancer, gastric tumors, bladder cancer, endometrial cancer, head and neck cancer, and renal cancer.

18. A method for detecting TIGIT, comprises: adding the expression cassette, recombinant vector or transgenic cell line according to claim 4 into a sample to be detected and waiting for binding to TIGIT in sample, then using enzyme-linked-immunosorbent serologic assay (ELISA) or flow cytometry (FCM) to detect.

19. A method for detecting TIGIT, comprises: adding the human TIGIT antibody or the antigen-binding fragment thereof according to claim 1 into a sample to be detected and waiting for binding to TIGIT in sample, then using enzyme-linked-immunosorbent serologic assay (ELISA) or flow cytometry (FCM) to detect.

20. A method for detecting TIGIT, comprises: adding the immunoconjugate according to claim 5 into a sample to be detected and waiting for binding to TIGIT in sample, then using enzyme-linked-immunosorbent serologic assay (ELISA) or flow cytometry (FCM) to detect.


SEQUENCE LISTING (26 sequences)

SEQ ID NO: 1 - protein, 115 aa, synthetic construct
EVQLQQSGPELVKPGASLKISCKTSGYTFTEYTMHWVKQSHGKSLEWIGGINPNNGGTKFKGKATLTVDKSSSTAYMELRSLTSEDSAVYYCARSGNWDYAMDYWGQGTSVTVSS

SEQ ID NO: 2 - protein, 8 aa, synthetic construct
GYTFTEYT

SEQ ID NO: 3 - protein, 8 aa, synthetic construct
INPNNGGT

SEQ ID NO: 4 - protein, 12 aa, synthetic construct
ARSGNWDYAMDY

SEQ ID NO: 5 - DNA, 345 bp, synthetic construct
  source 1..345: mol_type = other DNA
gaggtccagctgcaacaatctggacctgagctggtgaagcctggggcttcactgaagatatcctgcaagacttctggatacacattcactgaatacaccatgcactgggtgaagcagagccatggaaagagccttgagtggattggaggtattaatcctaacaatggtggtactaagttcaagggcaaggccacattgactgtagacaagtcctccagcacagcctacatggagctccgcagcctgacatctgaggattctgcagtctattactgtgcaagatcggggaactgggactatgctatggactactggggtcaaggaacctcagtcaccgtctcctca

SEQ ID NO: 6 - protein, 110 aa, synthetic construct
DIVMTQSHKFMSTSVGDRVSITCKASQHVSTAVVWYQQKPGQSPKLLIYSASYRYTGVDRFTGSGSGTDFTFTISSVQAEDLAVYYCQQHYITPWTFGGGTKLEIKRADA

SEQ ID NO: 7 - protein, 6 aa, synthetic construct
QHVSTA

SEQ ID NO: 8 - skipped

SEQ ID NO: 9 - protein, 9 aa, synthetic construct
QQHYITPWT

SEQ ID NO: 10 - DNA, 330 bp, synthetic construct
  source 1..330: mol_type = other DNA
gacattgtgatgacccagtctcacaaattcatgtccacatcagtaggagacagggtcagcatcacctgcaaggccagtcaacatgtgagtactgctgtagtctggtatcaacagaaaccaggacaatctcctaaactactgatttactcggcatcgtaccggtacactggagtccctgatcggttcactggcagtggatctgggacggatttcactttcaccatcagcagtgtgcaggctgaggacctggcagtttattactgtcagcaacattatattactccgtggacgttcggtggaggcaccaagctggaaatcaaacgggctgat

SEQ ID NO: 11 - protein, 121 aa, synthetic construct
QVQLQQSGAELVKPGASVKLSCKASGYTFTEYFIHWIKQKSGQGLEWIGWFYPGSGSIKYNERFKDKATLTADKSSSTVYMELSRLTSEDSAVYFCARHEMRYGNYVLDYWGQGTTLTVSS

SEQ ID NO: 12 - protein, 8 aa, synthetic construct
GYTFTEYF

SEQ ID NO: 13 - protein, 8 aa, synthetic construct
FYPGSGSI

SEQ ID NO: 14 - protein, 14 aa, synthetic construct
ARHEMRYGNYVLDY

SEQ ID NO: 15 - DNA, 363 bp, synthetic construct
  source 1..363: mol_type = other DNA
caggtccagctgcagcagtcaggagctgagctggtgaaacccggggcatcagtgaagctgtcctgtaaggcttctggctacaccttcactgagtattttatacactggataaagcagaagtctggacagggtcttgagtggattgggtggttttaccctggaagtggtagtataaagtacaatgagagattcaaggacaaggccacattgactgcggacaaatcctccagcacagtctatatggagcttagtagattgacatctgaagactctgcggtctatttctgtgcaagacacgagatgaggtatggtaactacgtccttgactactggggccaaggcaccactctcacagtctcctca

SEQ ID NO: 16 - protein, 113 aa, synthetic construct
QAVVTQESALTTSPGETVTLTCRSSTGAVTTRNYANWVQEKPDHLFTGLIGGTNNRVPGVPARFSGSLIGDKAALTITGAQTEDEAIYFCGLWYSNHLVFGGGTKLTVLGQPK

SEQ ID NO: 17 - protein, 9 aa, synthetic construct
TGAVTTRNY

SEQ ID NO: 18 - skipped

SEQ ID NO: 19 - protein, 9 aa, synthetic construct
GLWYSNHLV

SEQ ID NO: 20 - DNA, 339 bp, synthetic construct
  source 1..339: mol_type = other DNA
caggctgttgtgactcaggaatctgcactcaccacatcacctggtgaaacagtcacactcacttgtcgctcaagtactggggctgttacaactagaaactatgccaactgggtccaagaaaaaccagatcatttattcacaggtctaataggtggtaccaacaaccgagttccaggtgttcctgccagattctcaggctccctgattggagacaaggctgccctcaccatcacaggggcacagactgaggatgaggcaatttatttctgtggtctatggtacagcaaccatttggtgttcggtggaggaaccaaactgactgtcctaggccagcccaag

SEQ ID NO: 21 - protein, 119 aa, synthetic construct
QVQLVQSGAEVKKPGASVKVSCKTSGYTFTEYTMHWVRQAPGQRLEWIGGINPNNGGTSYNQKFQGRVTITVDTSASTAYMELSSLRSEDTAVYYCARSGNWDYAMDYWGQGTTVTVSS

SEQ ID NO: 22 - protein, 111 aa, synthetic construct
DIQMTQSPSSMSASVGDRVTITCKASQHVSTAVVWYQQKPGKAPKLLIYSASYRYTGVPDRFSGSGSGTDFTFTISSLQPEDIATYYCQQHYITPWTFGGGTKLEIKRTVA

SEQ ID NO: 23 - protein, 121 aa, synthetic construct
QVQLVQSGAEVKKPGASVKVSCKASGYTFTEYFIHWVRQAPGQGLEWIGWFYPGSGSIKYNERFKDRVTLTADTSISTAYMELSRLRSDDTAVYYCARHEMRYGNYVLDYWGQGTTVTVSS

SEQ ID NO: 24 - protein, 114 aa, synthetic construct
QAVVTQEPSLTVSPGGTVTLTCRSSTGAVTTRNYANWVQQKPGQAPRGLIGGTNNRVPGVPARFSGSLLGGKAALTLSGAQPEDEAEYYCGLWYSNHLVFGGGTKLTVLGQPKA

SEQ ID NO: 25 - protein, 577 aa, synthetic construct
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNKLEVLFQGPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 598 aa, synthetic construct
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNGPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK